SEQUENCE LISTING (59 sequences)

SEQ ID NO: 1 - protein, 17 aa, Unknown
  Signal Peptide of Cathepsin B (Cat)
MWWSLIPLSCLLALASA

SEQ ID NO: 2 - protein, 24 aa, Unknown
  Signal Peptide of C-C motif chemokine (CC)
MQFSARTLLCLLLTVAACSIYVLA

SEQ ID NO: 3 - protein, 24 aa, Unknown
  Signal Peptide of Nucleobindin-2 (Nuc)
MRWKIIQLQYCFLLVPCMLTALEA

SEQ ID NO: 4 - protein, 21 aa, Unknown
  Signal Peptide of Clusterin (Clus)
MKILLLCVGLLLTWDNGMVLG

SEQ ID NO: 5 - protein, 19 aa, Unknown
  Signal Peptide of Pigment epithelium-derived factor (Pig)
MQALVLLLWTGALLGHGSS

SEQ ID NO: 6 - protein, 24 aa, Unknown
  Signal Peptide of Procollagen C-endopeptidase enhancer 1 (Proco)
MLPAVLTSLLGPFLVAWVLPLARG

SEQ ID NO: 7 - protein, 31 aa, Unknown
  Signal Peptide of Sulfhydryl oxidase (Sulf)
MRRCGRHSGSPSQMLLLLLPPLLLAVPGAGA

SEQ ID NO: 8 - protein, 20 aa, Unknown
  Signal Peptide of Lipoprotein lipase (Lip)
MESKALLLVALGVWLQSLTA

SEQ ID NO: 9 - protein, 30 aa, Unknown
  Signal Peptide of Nidogen-1 (Nid)
MLDASGWKPAAWTWVLLLQLLLAGPGDCLS

SEQ ID NO: 10 - protein, 22 aa, Unknown
  Signal Peptide of Protein disulfide-isomerase (Pro)
MDDRLLTVLLLLLGVSGPWGQG

SEQ ID NO: 11 - DNA, 51 bp, Unknown
  gene of Signal Peptide of Cathepsin B (Cat)
atgtggtggtccttgattccgctctcttgcctgctggcactggcaagtgcc

SEQ ID NO: 12 - DNA, 72 bp, Unknown
  gene of Signal Peptide of C-C motif chemokine (CC)
atgcagttctccgcaagaacgcttctgtgcctgctactcacagttgctgcctgtagcatctatgtgctggcc

SEQ ID NO: 13 - DNA, 72 bp, Unknown
  gene of Signal Peptide of Nucleobindin-2 (Nuc)
atgaggtggaagatcatccagctacagtactgttttcttttggtcccgtgcatgcttactgctctggaagct

SEQ ID NO: 14 - DNA, 63 bp, Unknown
  gene of Signal Peptide of Clusterin (Clus)
atgaagattctcctgttgtgcgtggggctgctgctgacctgggacaatggcatggtcctggga

SEQ ID NO: 15 - DNA, 57 bp, Unknown
  gene of Signal Peptide of Pigment epithelium-derived factor (Pig)
atgcaggccctggtgctactcctctggacaggagccctgcttgggcatggcagcagc

SEQ ID NO: 16 - DNA, 72 bp, Unknown
  gene of Signal Peptide of Procollagen C-endopeptidase enhancer 1 (Proco)
atgctgcctgctgtcctaacctccctcctggggccattccttgtggcctgggtactgcctcttgcccgaggc

SEQ ID NO: 17 - DNA, 93 bp, Unknown
  gene of Signal Peptide of Sulfhydryl oxidase (Sulf)
atgaggaggtgcggccgccactcggggtcgccgtcgcagatgctactgctgctgctgccgccgctgctgctcgcggtgcccggcgctggcgcg

SEQ ID NO: 18 - DNA, 60 bp, Unknown
  gene of Signal Peptide of Lipoprotein lipase (Lip)
atggagagcaaagccctgctcctggtggctctgggagtgtggctccagagtttgaccgcc

SEQ ID NO: 19 - DNA, 90 bp, Unknown
  gene of Signal Peptide of Nidogen-1 (Nid)
atgctggacgcgagcggctggaagcccgcggcgtggacatgggtgctgctgctgcagctattgctggcggggcccggagactgcctgagc

SEQ ID NO: 20 - DNA, 66 bp, Unknown
  gene of Signal Peptide of Protein disulfide-isomerase (Pro)
atggatgatcggctcctgacagtgttgctgctcctgctgggtgtctcaggcccatggggacaggga

SEQ ID NO: 21 - protein, 339 aa, Unknown
  Cathepsin B (Cat)
MWWSLIPLSCLLALASAHNKPSFHPLSDDLINYINKRNTTWQAGRNFHNVDISYLKRLCGTIMGGPKLPERVAFAEDMELPENFDAREQWSNCPTIKQIRDQGSCGSCWAFGAVGAMSDRLCIHTNGHVNVEVSAEDLLTCCGSQCGDGCNGGYPSGAWNFWIKKGLVSGGLYNSHVGCLPYTIPPCEHHVNGSRPQCTGEGDTPKCTKSCEAGYSPSYKEDKHYGYTSYSVSNNEKEIMAEIYKNGPVEGAFTVFSDFLTYKSGVYKHEAGDIMGGHAIRILGWGVENSVPYWLVANSWNVDWGDNGLFKILRGEDHCGIESEIVAGIPRTDLYWGRF

SEQ ID NO: 22 - protein, 143 aa, Unknown
  C-C motif chemokine (CC)
MQFSARTLLCLLLTVAACSIYVLAQPDAVNSPLTCCYSFTAKRIPEKRLESYKRITSSKCPKEAVIFITKLKREICADPKQDWVQTYTKKLDQSQAKSEAATVYKTAPLNANLTHESAVNASTTAFPTTDLRTSVRVTSMTVN

SEQ ID NO: 23 - protein, 420 aa, Unknown
  Nucleobindin-2 (Nuc)
MRWKIIQLQYCFLLVPCMLTALEAVPIDVDKTKVHNTEPVESARIDPPDTGLYYDEYLKQVIDVLETDQHFREKLQKADIEEIRSGRLSKELDLVSHHVRTKLDELKRQEVARLRMLIKAKLDSLQDTGMNHHLLLKQFEHLNHQNPDTFESSDLDMLIKAATADLEQYDRTRHEEFKKYEMMKEHERREYLKTLNEEKRKEEESKFEEMKRKHENHPKVNHPGSKDQLKEVWEEADGLDPNDFDPKTFFKLHDVNNDGFLDEQELEALFTRELEKVYDPRNAEDDMIEMEEERLRMREHVMNEIDNNKDRLVTLEEFLRATEKKEFLEPDSWETLGQQQLFTEEELKEYESIIAMQENELKKRADELQKQKEELQRQHDHLEAQKQEYQQAVQQLEHKKFQQGIAPSGPAGELEFKPRM

SEQ ID NO: 24 - protein, 447 aa, Unknown
  Clusterin (Clus)
MKILLLCVGLLLTWDNGMVLGEQEVSDNELKEMSTQGSRYINKEIQNAVQGVKQIKTLIEKTNEERKSLLNSLEEAKKKKEDALDDTRDTEMKLKAFPEVCNETMMALWEECKPCLKQTCMKFYARVCRSGSGLVGRQLEEFLNQSSPFYFWMNGDRIDSLMESDRQQSQVLDAMQDSFTRASGIMDMLFQDRFFTHEPQDTHYFSPFGFPHRRPHFLYPKSRLVRSLIPLSHYGPPSFHDMFQPFLEMIHQAQQAMDVQFHRPAFQFPDKGLREGEDDRAVCKEIRHNSTGCLKMKGQCEKCQEILSVDCSANNPAQAHLRQELNDSLQMAERLTQQYNELLHSLQTKMLNTSSLLEQLNEQFNWVSQLANLTQGEDQYYLRVSTVTTHSNNSEEPSRVTEVVVKLFDSDPITVVLPEEVSKDNPKFMDTVAEKALQEYRKKSRAE

SEQ ID NO: 25 - protein, 378 aa, Unknown
  Pigment epithelium-derived factor (Pig)
MQALVLLLWTGALLGHGSSQNVASSSEEGSPAPDSTGEPVEEEEDPFFKVPVNKLAAAVSNFGYDLYRLRSSASPTANVLLSPLSVATALSALSLGAEQRTESIIHRALYYDLISNSDIHSTYKELLASVTAPEKSLKSASRIVFERKLRVRSSFVAPLEKSYGTRPRILTGNPRIDLQEINNWIQAQMKGKLARSTREMPSAISILLLGVAYFKGQWVTKFDSRKTSLQDFHLDEDRTVKVPMMSEPKAILRYGLDSDLNCKVWEHGGWEGSERGRVSSIRKSIWGYSKIHELQSLFESPDFSKITGKPVKLTQVEHRAAFEWNEEGVETSPNPGLQPVRLTFPLDYHLNQPFIFVLRDTDTGALLFIGKILDPRGT

SEQ ID NO: 26 - protein, 468 aa, Unknown
  Procollagen C-endopeptidase enhancer 1 (Proco)
MLPAVLTSLLGPFLVAWVLPLARGQTPNYTRPVFLCGGDVTGESGYVASEGFPNLYPPNKKCIWTITVPEGQTVSLSFRVFDMELHPSCRYDALEVFAGSGTSGQRLGRFCGTFRPAPVVAPGNQVTLRMTTDEGTGGRGFLLWYSGRATSGTEHQFCGGRMEKAQGTLTTPNWPESDYPPGISCSWHIIAPSDQVIMLTFGKFDVEPDTYCRYDSVSVFNGAVSDDSKRLGKFCGDKAPSPISSEGNELLVQFVSDLSVTADGFSASYKTLPRDAVEKELAPSPGEDVQLGPQSRSDPKTGTGPKVKPPSKPKFQPAEKPEVSPDTQETPVAPDPPSATCPKQYKRLGTLQSNFCASSLVVTGTVKTMVRGPGEGLTVTISLLGVYKSGGLDLPSPPTDTSLKLYVPCRQMPPMKKGASYLLMGQVEENRGPILPPESFLVPYKPNQDQILNNLRKRKCPSQPRPAA

SEQ ID NO: 27 - protein, 571 aa, Unknown
  Sulfhydryl oxidase (Sulf)
MRRCGRHSGSPSQMLLLLLPPLLLAVPGAGAVQVSVLYSSSDPVTVLNANTVRSTVLRSNGAWAVEFFASWCGHCIAFAPTWKELAYDVREWRPVLNLAVLDCAEETNTAVCRDFNISGFPTVRFFKAFSKNGSGITLPVADASVETLRRKLIDALESHSDMWSSSRPKLKPAKLVEINEFFAETNEDYLVLIFEDKDSYVGREVTLDLFQHHIPVHRVLNTERNAVSKFGVVEFPSCYLLFRNGSFSRVPVVMESRLFYTSYLKGMSGPILVDPPTTTISTDAPVTTDVVPTVWKVANHARIYMADLESSLHYIFLVEVGKFSVLEGQRLLALKKLVAVLAKYFPGRPLAQNFLHSIHDWLQRQQRKKIPYKFFRAALDNRKEGIVLTEKVNWVGCQGSKPHFRGFPCSLWILFHFLTVQASRYSENHPQEPADGQEVLQAMRSYVQWFFGCRDCAEHFENMAASTMHRVRSPTSAVLWLWTSHNKVNARLSGAPSEDPYFPKVQWPLRELCFDCHNEINGREPVWDLEATYRFLKAHFSSENIILDTPVAGLATQRNPQILGATPEPHM

SEQ ID NO: 28 - protein, 473 aa, Unknown
  Lipoprotein lipase (Lip)
  MISC_FEATURE (24)..(24): x is any amino acid
MESKALLLVALGVWLQSLTASQGXAAADGGRDFTDIESKFALRTPDDTAEDNCHLIPGIAESVSNCHFNHSSKTFVVIHGWTVTGMYESWVPKLVAALYKREPDSNVIVVDWLYRAQQHYPVSAGYTKLVGNDVARFINWMEEEFNYPLDNVHLLGYSLGAHAAGVAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCNSKEAFEKGLCLSCRKNRCNNVGYEINKVRAKRSSKMYLKTRSQMPYKVFHYQVKIHFSGTESDKQLNQAFEISLYGTVAESENIPFTLPEVSTNKTYSFLIYTEVDIGELLMMKLKWKSDSYFSWSDWWSSPGFVIEKIRVKAGETQKKVIFCAREKVSHLQKGKDSAVFVKCHDKSLKKSG

SEQ ID NO: 29 - protein, 1212 aa, Unknown
  Nidogen-1 (Nid)
MLDASGWKPAAWTWVLLLQLLLAGPGDCLSRQELFPFGPGQGDLELEAGDDVVSPALELIGELSFYDRSDITSVYVTTNGIIAMSEPPARESHPGTFPPSFGSVAPFLADLDTTDGLGNVYYREDLSPSIMQMAAEYVQRGFPEVPFQPTSVVVVTWESVAPYEGPSGSSAQEGKRNTFQAVLASSNSSSYAIFLYPEDGLQFFTTFSKKDENQVPAMVGFSQGLVGFLWRSDGAYNIFANDRESIENLAKSSNAGHQGVWVFEIGSPATAKGVVSADVNLGLDDDGSDYEDEEYDLATSHLGLEDMATQPFPSPSPRRGNTHPHDVPRVLSPSYEATERPHGIPTERTRTFQLPAERFHQQHPQVIDVDEVEETGIVFSYNIGSQQTCANNRHQCSVHAECRDYATGFCCRCVANYTGNGRQCVAEGSPQRVNGKVKGRIFVGNSQVPVVFENTDLHSYVVMNHGRSYTAISTIPETVGYSLLPLAPIGGIIGWMFAVEQNGFKNGFSITGGEFTRQAEVTFLGHPGKLVLKQHFSGIDEHGHLTINTELDGRVPQIPYGSSVHIEPYTELYHYSSSVITSSSTREYTVTEPDPDGTAPSHTHVYQWRQTITFQECVHDDSRPALPSTQQLSVDSVFVLYNQEERILRYALSNSIGPVREGSPDALQNPCYIGTHGCDSNAACRPGPGTQFTCECSIGFRGDGQTCYDIDECSEQPSRCGNHAACNNSPGAYLCECVEGYHFSDGGICVADVDQRPINYCETGLHNCDIPQRAQCIYMGGSSYTCSCLPGFSGDGRACQDVDECQLSRCHPDAFCYNTPGSFTCQCKPGYQGDGFQCVPGEVGKTRCQLEREHILGASGVADAQQPRLLGMYVPQCDEYGHYEPTQCHHGTGYCWCVDRDGRELEGTRTQPGMRPPCLSTVAPPIHQRPVVPTAVIPLPPGTHLLFAQTGKIERLPLEGNTMKKTEAKAFFHIPAKVIIGLAFDCVDKVVYWTDISEPSIGRASLHGGEPTTIIRQDLGSPEGIALDHLGRNIFWTDSQLDRIEVARMDGTQRRVLFDTGLVNPRGIVTDSVGGNLYWTDWNRENPKIETSYMDGTNRRILAQDNLGLPNGLTFDAFSSQLCWVDAGTHRAECLNPAQPSSRKVLEGLQYPFAVTSYGKNLYYTDWKTNSVIAMDLAISKEMDAFTPTSRPGYMASPLPCPNALKATTTAQ

SEQ ID NO: 30 - protein, 527 aa, Unknown
  Protein disulfide-isomerase (Pro)
MDDRLLTVLLLLLGVSGPWGQGQEPEGPSEVLPEESSGEEVPKEDGILVLSHHTLSLALQEHPALMVEFYAPWCGHCKALAPEYSKAAALLAAESASVTLAKVDGPAEPELTKEFGVVGYPTLKFFQNGNRTNPEEYTGPQKAEGIAEWLRRRVGPSAKRLEDEEDVQALTDKWEVVVIGFFQDLQGEDVATFLALARDALDITFGFTDQPQLFQKFGLTKDTVILFKKFDEGRADFPVDKDTGLDLGDLSRFLVTHSMHLVTEFNSQTSPKIFAAKILNHLLLFVNKTLAQHRELLTDFREAAPPFRGQVLFVMVDVAADNDHVLNYFGLKAEEAPTLRLINVETTKKYAPTGLVPITAASVAAFCQAVLHGQVKPYLLSQEIPPDWDERPVKTLVGKNFEQVAFDETKNVFVKFYAPWCSHCKEMAPAWEALAEKYRDREDIVIAELDATANELEAFSVHGYPTLKFFPAGPDRKVIEYKSTRDLETFSKFLDSGGNLPEEEPKEPAISTPEIQDNSTVGPKEEL

SEQ ID NO: 31 - protein, 236 aa, Unknown
  mCherry
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 32 - DNA, 711 bp, Unknown
  gene of mCherry
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 33 - protein, 22 aa, Unknown
  SP7.2
MHRPEAMLLLLTLALLGGPTWA

SEQ ID NO: 34 - DNA, 66 bp, Unknown
  gene of SP7.2
atgcaccggccagaggccatgctgctgctgctcacgcttgccctcctggggggccccacctgggca

SEQ ID NO: 35 - DNA, 88 bp, Artificial Sequence
  Cat_SP_F
agggggtaccgccaccatgtggtggtccttgattccgctctcttgcctgctggcactggcaagtgccgtgagcaagggcgaggaggat

SEQ ID NO: 36 - DNA, 54 bp, Artificial Sequence
  CC_SP_F1
acagttgctgcctgtagcatctatgtgctggccgtgagcaagggcgaggaggat

SEQ ID NO: 37 - DNA, 78 bp, Artificial Sequence
  CC_SP_F2
agggggtaccgccaccatgcagttctccgcaagaacgcttctgtgcctgctactcacagttgctgcctgtagcatcta

SEQ ID NO: 38 - DNA, 52 bp, Artificial Sequence
  Nuc_SP_F1
ggtcccgtgcatgcttactgctctggaagctgtgagcaagggcgaggaggat

SEQ ID NO: 39 - DNA, 81 bp, Artificial Sequence
  Nuc_SP_F2
agggggtaccgccaccatgaggtggaagatcatccagctacagtactgttttcttttggtcccgtgcatgcttactgctct

SEQ ID NO: 40 - DNA, 60 bp, Artificial Sequence
  Clus_SP_F1
gggctgctgctgacctgggacaatggcatggtcctgggagtgagcaagggcgaggaggat

SEQ ID NO: 41 - DNA, 65 bp, Artificial Sequence
  Clus_SP_F2
agggggtaccgccaccatgaagattctcctgttgtgcgtggggctgctgctgacctgggacaatg

SEQ ID NO: 42 - DNA, 90 bp, Artificial Sequence
  Pig_SP_F
ggtaccgccaccatgcaggccctggtgctactcctctggacaggagccctgcttgggcatggcagcagcgtgagcaagggcgaggaggat

SEQ ID NO: 43 - DNA, 55 bp, Artificial Sequence
  Proco_SP_F1
ccttgtggcctgggtactgcctcttgcccgaggcgtgagcaagggcgaggaggat

SEQ ID NO: 44 - DNA, 76 bp, Artificial Sequence
  Proco_SP_F2
agggggtaccgccaccatgctgcctgctgtcctaacctccctcctggggccattccttgtggcctgggtactgcct

SEQ ID NO: 45 - DNA, 75 bp, Artificial Sequence
  Sulf_SP_F1
atgctactgctgctgctgccgccgctgctgctcgcggtgcccggcgctggcgcggtgagcaagggcgaggaggat

SEQ ID NO: 46 - DNA, 81 bp, Artificial Sequence
  Sulf_SP_F2
agggggtaccgccaccatgaggaggtgcggccgccactcggggtcgccgtcgcagatgctactgctgctgctgccgccgct

SEQ ID NO: 47 - DNA, 58 bp, Artificial Sequence
  Lip_SP_F1
ggtggctctgggagtgtggctccagagtttgaccgccgtgagcaagggcgaggaggat

SEQ ID NO: 48 - DNA, 62 bp, Artificial Sequence
  Lip_SP_F2
agggggtaccgccaccatggagagcaaagccctgctcctggtggctctgggagtgtggctcc

SEQ ID NO: 49 - DNA, 72 bp, Artificial Sequence
  Nid_SP_F1
gggtgctgctgctgcagctattgctggcggggcccggagactgcctgagccgtgagcaagggcgaggaggat

SEQ ID NO: 50 - DNA, 83 bp, Artificial Sequence
  Nid_SP_F2
agggggtaccgccaccatgctggacgcgagcggctggaagcccgcggcgtggacatgggtgctgctgctgcagctattgctgg

SEQ ID NO: 51 - DNA, 65 bp, Artificial Sequence
  Pro_SP_F1
tgttgctgctcctgctgggtgtctcaggcccatggggacagggagtgagcaagggcgaggaggat

SEQ ID NO: 52 - DNA, 65 bp, Artificial Sequence
  Pro_SP_F2
agggggtaccgccaccatggatgatcggctcctgacagtgttgctgctcctgctgggtgtctcag

SEQ ID NO: 53 - DNA, 36 bp, Artificial Sequence
  mCherry XhoR
agggctcgagtttacttgtacagctcgtccatgccg

SEQ ID NO: 54 - DNA, 36 bp, Artificial Sequence
  Bgl_Hprt 5'F
agggagatctcaccagttttcattttctgtgtgaga

SEQ ID NO: 55 - DNA, 42 bp, Artificial Sequence
  Hprt 5'_NruR
agggtcgcgaattaggaatacaaaataaatctaggtcatagc

SEQ ID NO: 56 - DNA, 35 bp, Artificial Sequence
  Sal_Hprt 3'F
aggggtcgaccactatgtcgaggatttggaaaagg

SEQ ID NO: 57 - DNA, 34 bp, Artificial Sequence
  Hprt 3'_SalR
aggggtcgacgaggatgggagtgagagtgaactg

SEQ ID NO: 58 - protein, 218 aa, Unknown
  Light chain of Pembrolizumab
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 447 aa, Unknown
  Heavy chain of Pembrolizumab
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2023/0234995 A1
(43) Pub. Date: Jul. 27, 2023

(54) CHO CELL-DERIVED PROTEIN SECRETORY FACTORS AND EXPRESSION VECTORS COMPRISING THE SAME

(71) Applicant: LG CHEM, LTD, Seoul (KR)

(72) Inventors: Byeong Eun MIN, Seoul (KR); Yeonchul KIM, Seoul (KR); Young Sam PARK, Seoul (KR); Saem JUNG, Seoul (KR)

(73) Assignee: LG CHEM, LTD, Seoul (KR)

(21) Appl. No.: 17/781,873

(22) PCT Filed: Dec. 1, 2020
(86) PCT No.: PCT/KR2020/017413

§ 371 Date: Jun. 2, 2022

(30) Foreign Application Priority Data
Dec. 2, 2019 (KR) 10-2019-0158447

(51) Int. Cl. C07K 14/47; C12N 15/90; C12N 5/00; C12N 9/64; C12N 9/02; C12N 9/20; C12N 9/90; C07K 14/52; C07K 14/81; C07K 14/78; C07K 14/435

(52) U.S. Cl. CPC C07K 14/47; C07K 14/78; C07K 14/81; C07K 14/523; C07K 14/43595; C12N 5/00; C12N 9/20; C12N 9/0051; C12N 9/90; C12N 9/6472; C12N 15/907; C12Y 108/03002; C12Y 301/01034; C12Y 304/22001; C12Y 503/04001

(57) ABSTRACT

The present invention relates to a CHO cell-derived protein secretory factor, an expression cassette in which a nucleic acid sequence encoding the protein secretory factor; and a gene encoding a target protein are operably linked, an expression vector comprising the expression cassette, a transformed cell into which the expression vector is introduced, and a method for producing a target protein using the transformed cell.

### CROSS REFERENCE TO RELATED APPLICATION(S)

This application is a 35 U.S.C. 371 National Phase Entry Application from PCT/KR2020/017413 filed on Dec. 1, 2020, which claims priority to and the benefits of Korean Patent Application No. 10-2019-0158447, filed on Dec. 2, 2019, the entire contents of which are incorporated herein by reference.

The instant application contains a Sequence Listing which has been submitted via EFS-Web and is hereby incorporated by reference in its entirety. Said Sequence Listing, created on Nov. 28, 2022, is named 3570-823_ST25.txt and is 64,709 bytes in size.

### TECHNICAL FIELD

The present invention relates to a CHO cell-derived protein secretory factor, an expression cassette in which a nucleic acid sequence encoding the protein secretory factor; and a gene encoding a target protein are operably linked, an expression vector including the expression cassette, a transformed cell into which the expression vector is introduced, and a method for producing a target protein using the transformed cell.

### BACKGROUND ART

Recombinant proteins can be produced on a large-scale using microbial or animal cell systems through genetic recombination technology for useful protein components that are difficult to obtain in vivo. Recombinant proteins can be regulated such that they can be expressed in cells or secreted outside the cells. However, the intracellular expression has a disadvantage in that the proteins are often accumulated as an insoluble mass, and that productivity is lowered due to difficulty in separation and purification. On the other hand, soluble proteins with correct protein folding can be easily obtained through extracellular secretion. Thus, for more suitable extracellular secretion in terms of protein production yield and quality control, an optimized recombinant protein expression system is important.

In order to produce a recombinant protein, components such as a host cell, a gene of interest, an expression vector, a selective marker, a promoter, and a signal peptide sequence are essentially required. The quality and productivity of the recombinant protein vary depending on the selection of these components.

In the case of the signal peptide, it is located at the N-terminal region of the recombinant protein to be produced and thus is involved in the expression level of the recombinant protein, and is a component that allows the extracellular secretion. Depending on which signal peptide is used, a difference in expression level can be observed, and the signal peptide sequence may remain at the N-terminus of the protein due to the mis-cleavage of the signal peptide, affecting the quality of the recombinant protein.

Therefore, it is important to select a signal peptide that does not cause mis-cleavage and can induce high expression.

Meanwhile, conventional signal peptides mostly use human-derived signal peptides, and CHO cells are mainly used as expression host cells. Signal peptides between host cells may be used in combination, but may cause problems in terms of quality.

Under these circumstances, the present inventors have made extensive efforts to increase the expression level in CHO cells and solve the mis-cleavage problem. As a result, they have developed a novel signal peptide, which is a polypeptide consisting of 17 to 31 amino acid sequences derived from CHO cells, and have completed the present invention by confirming that the signal peptide can be significantly increased in terms of expression and cleaved 100% at a cleavage site to prevent mis-cleavage.

### DISCLOSURE

### Technical Problem

An object of the present invention is to provide a protein secretory factor consisting of an amino acid sequence of SEQ ID NO: 1, SEQ ID NO: 2, or SEQ ID NO: 3.

Another object of the present invention is to provide an expression cassette in which a nucleic acid sequence encoding a protein secretory factor consisting of an amino acid sequence of SEQ ID NO: 1, SEQ ID NO: 2, SEQ ID NO: 3, SEQ ID NO: 4, or SEQ ID NO: 5; and a gene encoding a target protein are operably linked.

Still another object of the present invention is to provide an expression vector for secreting a target protein, including an expression cassette in which a nucleic acid sequence encoding a protein secretory factor consisting of an amino acid sequence of SEQ ID NO: 1, SEQ ID NO: 2, SEQ ID NO: 3, SEQ ID NO: 4, or SEQ ID NO: 5; and a gene encoding a target protein are operably linked.

Still another object of the present invention is to provide a transformed cell in which the expression vector is introduced into a host cell.

Still another object of the present invention is to provide a method for producing a target protein, including:
i) culturing a transformed cell including an expression vector for secreting a target protein, which includes an expression cassette in which a nucleic acid sequence encoding a protein secretory factor consisting of an amino acid sequence of SEQ ID NO: 1, SEQ ID NO: 2, SEQ ID NO: 3, SEQ ID NO: 4, or SEQ ID NO: 5; and a gene encoding a target protein are operably linked; and
ii) recovering the target protein from the culture medium or culture supernatant of the cultured cells.

### Technical Solution

Hereinbelow, the present invention will be described in detail. Meanwhile, each of the explanations and exemplary embodiments disclosed herein can be applied to other explanations and exemplary embodiments. That is, all combinations of various factors disclosed herein belong to the scope of the present invention. Furthermore, the scope of the present invention should not be limited by the specific disclosure provided hereinbelow.

Additionally, those of ordinary skill in the art may be able to recognize or confirm, using only conventional experimentation, many equivalents to the particular aspects of the invention described herein. Furthermore, it is also intended that these equivalents be included in the present invention.

In order to achieve the objects above, one aspect of the present invention provides a novel protein secretory factor derived from CHO cells. Specifically, the present invention provides a protein secretory factor consisting of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10. More specifically, the protein secretory factor may consist of an amino acid sequence of SEQ ID NO: 1, SEQ ID NO: 2, or SEQ ID NO: 3, but is not limited thereto.

The “protein secretory factor” of the present invention refers to a factor that is linked to a target protein to induce the extracellular secretion of the target protein, and may consist of a polypeptide. The protein secretory factor may promote the secretion of a target protein, that is, an endogenous protein and/or a foreign protein, and in particular, may promote the extracellular secretion of the light and/or heavy chain of an antibody, but is not limited thereto.

The protein secretory factor in the present invention may be interchangeably used with “signal sequence” or “signal peptide (SP)”.

The protein secretory factor of the present invention may have an amino acid sequence of SEQ ID NO: 1, SEQ ID NO: 2, or SEQ ID NO: 3, but is not limited thereto. Additionally, the protein secretory factor of the present invention may further include a protein secretory factor consisting of an amino acid sequence of SEQ ID NO: 4, SEQ ID NO: 5, SEQ ID NO: 6, SEQ ID NO: 7, SEQ ID NO: 8, SEQ ID NO: 9, or SEQ ID NO: 10, but is not limited thereto.

In one specific embodiment of the present invention, the protein secretory factor may be derived from CHO cells, but is not limited thereto. As used herein, the term “CHO cell” is a Chinese hamster ovary cell, and may be a host cell for transformation commonly used in the art. In addition, a CHO-cell derived protein secretory factor may be selected to increase the expression level in CHO cells, which are host cells.

In the present invention, the protein secretory factors consisting of the amino acid sequence of SEQ ID NO: 1 may be cathepsin B (Cat), and may be used interchangeably with the Cat secretion sequence in the present invention. The protein secretory factor consisting of the amino acid sequence of SEQ ID NO: 2 may be a C—C motif chemokine (CC), and may be used interchangeably with the CC secretion sequence in the present invention. The protein secretory factor consisting of the amino acid sequence of SEQ ID NO: 3 may be nucleobindin-2 (NUC), and may be used interchangeably with the NUC secretion sequence in the present invention.

Additionally, the protein secretory factor consisting of the amino acid sequence of SEQ ID NO: 4 of the present invention may be clusterin (Clus), and may be used interchangeably with the Clus secretion sequence in the present invention. The protein secretory factor consisting of the amino acid sequence of SEQ ID NO: 5 may be a pigment epithelium-derived factor (Pig), and may be used interchangeably with the Pig secretion sequence in the present invention. The protein secretory factor consisting of the amino acid sequence of SEQ ID NO: 6 may be procollagen C-endopeptidase enhancer 1 (Proco), and may be used interchangeably with the Proco secretion sequence in the present invention. The protein secretory factor consisting of the amino acid sequence of SEQ ID NO: 7 may be sulfhydryl oxidase (Sulf), and may be used interchangeably with the Sulf secretion sequence in the present invention. The protein secretory factor consisting of the amino acid sequence of SEQ ID NO: 8 may be lipoprotein lipase (Lip), and may be used interchangeably with the Lip secretion sequence in the present invention. The protein secretory factor consisting of the amino acid sequence of SEQ ID NO: 9 may be nidogen-1 (Nid), and may be used interchangeably with the Nid secretion sequence in the present invention. The protein secretory factor consisting of the amino acid sequence of SEQ ID NO: 10 may be protein disulfide-isomerase (Pro), and may be used interchangeably with the Pro secretion sequence in the present invention.

The nucleic acid sequence encoding the cathepsin B signal peptide consisting of the amino acid sequence of SEQ ID NO: 1 may be a polynucleotide sequence of SEQ ID NO: 11, the nucleic acid sequence encoding the C—C motif chemokine signal peptide consisting of the amino acid sequence of SEQ ID NO: 2 may be a polynucleotide sequence of SEQ ID NO: 12, and the nucleic acid sequence encoding the nucleobindin-2 signal peptide consisting of the amino acid sequence of SEQ ID NO: 3 may be a polynucleotide sequence of SEQ ID NO: 13.

Additionally, the nucleic acid sequence encoding the clusterin signal peptide consisting of the amino acid sequence of SEQ ID NO: 4 may be a polynucleotide sequence of SEQ ID NO: 14, the nucleic acid sequence encoding the pigment epithelium-derived factor (Pig) signal peptide consisting of the amino acid sequence of SEQ ID NO: 5 may be a polynucleotide sequence of SEQ ID NO: 15, the nucleic acid sequence encoding the procollagen C-endopeptidase enhancer 1 (Proco) signal peptide consisting of the amino acid sequence of SEQ ID NO: 6 may be a polynucleotide sequence of SEQ ID NO: 16, the nucleic acid sequence encoding the sulfhydryl oxidase (Sulf) signal peptide consisting of the amino acid sequence of SEQ ID NO: 7 may be a polynucleotide sequence of SEQ ID NO: 17, the nucleic acid sequence encoding the lipoprotein lipase (Lip) signal peptide consisting of the amino acid sequence of SEQ ID NO: 8 may be a polynucleotide sequence of SEQ ID NO: 18, the nucleic acid sequence encoding the nidogen-1 (Nid) signal peptide consisting of the amino acid sequence of SEQ ID NO: 9 may be a polynucleotide sequence of SEQ ID NO: 19, and the nucleic acid sequence encoding the protein disulfide-isomerase (Pro) signal peptide consisting of the amino acid sequence of SEQ ID NO: 10 may be a polynucleotide sequence of SEQ ID NO: 20.

Although the protein secretory factor of the present invention is described as “a secretory factor consisting of a specific amino acid sequence”, it is apparent that as long as the secretory factor has an activity identical or corresponding to that of a secretory factor consisting of an amino acid sequence of the corresponding sequence number, it does not exclude a mutation that may occur by a meaningless sequence addition upstream or downstream of the amino acid sequence, a mutation that may occur naturally, or a silent mutation thereof. Even when the sequence addition or mutation is present, it falls within the scope of the present invention.

For example, as long as the secretory factor can function as a signal peptide identically or correspondingly to the nucleic acid molecules consisting of the polynucleotides, nucleic acid sequences showing a homology and/or identity of 85% or more, specifically 90% or more, more specifically 95% or more, even more specifically 98% or more, or even more specifically 99% or more to the sequence above can also be included in the present invention without limitation. Additionally, it is obvious that a nucleic acid sequence with deletion, modification, substitution, or addition in part of the sequence also can be included in the scope of the present invention, as long as the nucleic acid sequence has such homology.

As used herein, the term “homology” or “identity” refers to a degree of relevance between two given amino acid sequences or nucleic acid sequences, and may be expressed as a percentage. The terms “homology” and “identity” may often be used interchangeably with each other.

The sequence homology or identity of conserved polynucleotide or polypeptide sequences may be determined by standard alignment algorithms and can be used with a default gap penalty established by the program being used. Substantially, homologous or identical sequences are generally expected to hybridize to all or at least about 50%, 60%, 70%, 80%, or 90% or more of the entire length of the sequences under moderate or high stringent conditions. Polynucleotides that contain degenerate codons instead of codons in the hybridizing polypeptides are also considered.

Whether any two polynucleotide or polypeptide sequences have a homology, similarity, or identity may be determined by a known computer algorithm such as the “FASTA” program (Pearson et al., (1988) [Proc. Natl. Acad. Sci. USA 85]: 2444) using default parameters. Alternatively, it may be determined by the Needleman-Wunsch algorithm (Needleman and Wunsch, 1970, J. Mol. Biol. 48: 443-453), which is performed using the Needleman program of the EMBOSS package (EMBOSS: The European Molecular Biology Open Software Suite, Rice et al., 2000, Trends Genet. 16: 276-277) (preferably, version 5.0.0 or versions thereafter) (GCG program package (Devereux, J., et al., Nucleic Acids Research 12: 387 (1984)), BLASTP, BLASTN, FASTA (Atschul, [S.] [F.,] [ET AL., J MOLEC BIOL 215]: 403 (1990); Guide to Huge Computers, Martin J. Bishop, [ED.,] Academic Press, San Diego, 1994, and [CARILLO ETA/.](1988) SIAM J Applied Math 48: 1073). For example, the homology, similarity, or identity may be determined using BLAST or ClustalW of the National Center for Biotechnology Information (NCBI).

The homology, similarity, or identity of polynucleotides or polypeptides may be determined by comparing sequence information using, for example, the GAP computer program, such as Needleman et al. (1970), J Mol Biol. 48: 443 as disclosed in Smith and Waterman, Adv. Appl. Math (1981) 2:482. In summary, the GAP program defines the homology, similarity, or identity as the value obtained by dividing the number of similarly aligned symbols (i.e. nucleotides or amino acids) by the total number of the symbols in the shorter of the two sequences. Default parameters for the GAP program may include (1) a unary comparison matrix (containing a value of 1 for identities and 0 for non-identities) and the weighted comparison matrix of Gribskov et al. (1986), Nucl. Acids Res. 14:6745, as disclosed in Schwartz and Dayhoff, eds., Atlas of Protein Sequence and Structure, National Biomedical Research Foundation, pp. 353-358 (1979) (or EDNAFULL substitution matrix (EMBOSS version of NCBI NUC4.4)); (2) a penalty of 3.0 for each gap and an additional 0.10 penalty for each symbol in each gap (or a gap opening penalty of 10 and a gap extension penalty of 0.5); and (3) no penalty for end gaps. Therefore, as used herein, the term “homology” or “identity” refers to the relevance between sequences.

Another aspect of the present invention provides an expression cassette in which a nucleic acid sequence encoding a protein secretory factor consisting of an amino acid sequence of SEQ ID NO: 1, SEQ ID NO: 2, SEQ ID NO: 3, SEQ ID NO: 4, or SEQ ID NO: 5; and a gene encoding a target protein are operably linked

The “protein secretory factor” of the present invention is as described above.

As used herein, the term “target protein” may refer to a protein endogenously expressed in a host cell or a protein expressed by a foreign gene introduced thereinto. The type of target protein is not particularly limited as long as extracellular secretion efficiency is increased by the signal peptide sequence of the present invention.

The target protein may be antibody, antibody fragment (Fab or ScFv), fusion protein, protein scaffold, human growth hormone, serum protein, immunoglobulin, cytokine, α-, β- or γ-interferon, granulocyte-macrophage colony-stimulating factor (GM-CSF), platelet-derived growth factor (PDGF), phospholipase-activating protein (PLAP), insulin, tumor necrosis factor (TNF), growth factor, hormone, calcitonin, calcitonin gene-related peptide (CGRP), enkephalin, somatomedin, erythropoietin, hypothalamic-releasing factor, growth differentiation factor, cell adhesion protein, prolactin, chorionic gonadotropin, tissue plasminogen activator, growth hormone releasing peptide (GHPR), thymic humoral factor (THF), asparaginase, arginase, arginine deaminase, adenosine deaminase, peroxide dismutase, endotoxinase, catalase, chymotrypsin, lipase, uricase, adenosine diphosphatase, tyrosinase, bilirubin oxidase, glucose oxidase, glucodase, galactosidase, glucocerebrosidase, or glucuronidase, and specifically, it may be the heavy chain protein or the light chain protein of an antibody, but is not limited thereto.

As used herein, the term “operably linked” refers to a functional linkage between the above gene sequence, a promoter sequence, and a signal peptide sequence to initiate and mediate the transcription of the nucleic acid sequence encoding the protein secretory factor of the present application and the gene encoding the target protein. The operable linkage may be prepared using a gene recombination technique known in the art, and the site-specific DNA linkage may be prepared using a linking enzyme known in the art, but is not limited thereto

As used herein, the term “expression cassette” refers to a sequence regulating one or more genes and expression thereof, for example, a nucleic acid sequence including any combination of various cis-acting transcription regulating elements. The expression cassette of the present invention may further include various elements, for example, nucleic acid sequences such as a promoter and an enhancer, which are recognized in the art to be necessary for expression regulation, as well as the nucleic acid sequence encoding the protein secretion factor and the target protein. The sequence regulating the expression of a gene, that is, the sequence regulating the transcription of a gene and the expression of the transcription product thereof, is generally referred to as a “regulatory unit”. Most of the regulatory unit is located upstream of a coding sequence of a target gene such that it is operably linked thereto. In addition, the expression cassette may include a 3′ non-transcriptional region including a poly-adenylation site at a 3′ terminal.

The expression cassette of the present invention may be a combination of polynucleotides, which allows the extracellular secretion and expression of target proteins in a host cell, by operably linking the nucleic acid sequence encoding the protein secretory factor consisting of the amino acid sequence of SEQ ID NO: 1, SEQ ID NO: 2, SEQ ID NO: 3, SEQ ID NO: 4, or SEQ ID NO: 5; and the gene encoding the target protein.

Still another aspect of the present invention provides an expression cassette in which a gene encoding a target protein is operably linked to a nucleic acid sequence encoding a protein secretory factor consisting of an amino acid sequence of SEQ ID NO: 11 to SEQ ID NO: 20, and.

The “protein secretory factor”, “target protein”, “operably linked”, and “expression cassette” of the present invention are as described above.

In the present invention, the protein secretory factor encoded by the polynucleotide sequence of SEQ ID NO: 11 may be cathepsin B (Cat), the protein secretory factor encoded by the polynucleotide sequence of SEQ ID NO: 12 may be a C—C motif chemokine (CC), the protein secretory factor encoded by the polynucleotide sequence of SEQ ID NO: 13 may be nucleobindin-2 (Nuc), the protein secretory factor encoded by the polynucleotide sequence of SEQ ID NO: 14 may be clusterin (Clus), the protein secretory factor encoded by the polynucleotide sequence of SEQ ID NO: 15 may be a pigment epithelium-derived factor (Pig), the protein secretory factor encoded by the polynucleotide sequence of SEQ ID NO: 16 may be procollagen C-endopeptidase enhancer 1 (Proco), the protein secretory factor encoded by the polynucleotide sequence of SEQ ID NO: 17 may be sulfhydryl oxidase (Sulf), the protein secretory factor encoded by the polynucleotide sequence of SEQ ID NO: 18 may be lipoprotein lipase (Lip), the protein secretory factor encoded by the polynucleotide sequence of SEQ ID NO: 19 may be nidogen-1 (Nid), and the protein secretory factor encoded by the polynucleotide sequence of SEQ ID NO: 20 may be protein disulfide-isomerase (Pro).

Still another aspect of the present invention provides an expression vector for secreting a target protein, including an expression cassette in which a nucleic acid sequence encoding a protein secretory factor consisting of an amino acid sequence of SEQ ID NO: 1, SEQ ID NO: 2, SEQ ID NO: 3, SEQ ID NO: 4, or SEQ ID NO: 5; and a gene encoding a target protein are operably linked.

The “protein secretory factor”, “target protein”, “operably linked”, and “expression cassette” of the present invention are as described above.

As used herein, the term “expression vector for secreting a target protein” refers to an expression vector, in which the protein secretory factor and the gene encoding the target protein are operably linked to induce the extracellular secretion of the target protein when the vector is introduced into a host cell and expressed therein.

As used herein, the term “expression vector” generally refers to a double-stranded DNA fragment as a carrier into which a target DNA fragment encoding a target protein is inserted. The expression vector used in expressing a protein in the art may be used without limitation. Once the expression vector is in a host cell, the expression vector can be replicated regardless of a host chromosomal DNA, and the inserted target DNA can be expressed. In order to increase the expression level of a transfected gene in a host cell, the transfected gene must be operably linked to transcription and translation control sequences which are operated in a selected expression host cell.

The expression vector used in the present invention is not particularly limited as long as it can be replicated in a host cell, and any vector known in the art may be used. Examples of conventionally used vectors may include natural or recombinant plasmids, cosmids, viruses, and bacteriophages. For example, as a phage vector or cosmid vector, pWE15, M13, λMBL3, λMBL4, λIXII, λASHII, λAPII, λt10, λt11, Charon4A, and Charon21A, etc. may be used, and as a plasmid vector, those based on pBR, pUC, pBluescriptll, pGEM, pTZ, pCL, pET, etc. may be used. Specifically, the vector may be those based on pTZ, but is not limited thereto.

In one specific embodiment of the present invention, an expression vector for secreting a target protein was prepared by operably linking the nucleic acid sequence encoding the protein secretory factor consisting of the amino acid sequence of SEQ ID NO: 1, SEQ ID NO: 2, or SEQ ID NO: 3 and the gene encoding the target protein, based on the pTz-D1G1 vector (a variant including the promoter of Korean Patent No. 10-1038126) (Example 4).

The expression vector may further include a nucleic acid sequence encoding a protein secretory factor consisting of an amino acid sequence of SEQ ID NO: 6, SEQ ID NO: 7, SEQ ID NO: 8, SEQ ID NO: 9, or SEQ ID NO: 10.

Still another aspect of the present invention provides a transformed cell in which the expression vector is introduced into a host cell.

The “expression vector” of the present invention is as described above.

As used herein, the term “transformation” refers to a process of introducing a vector including a polynucleotide encoding a target polypeptide into a host cell, thereby allowing the expression of the protein encoded by the polynucleotide in the host cell.

As long as the transformed polynucleotide can be expressed in a host cell, it does not matter whether it is inserted into the chromosome of a host cell and located therein, or located outside the chromosome, and both cases may be included. Additionally, the polynucleotide includes DNA and RNA which encode the target polypeptide. The polynucleotide may be introduced in any form as long as it can be introduced into a host cell and expressed therein. For example, the polynucleotide may be introduced into a host cell in the form of an expression cassette, which is a gene construct including all elements necessary for self-expression. The expression cassette may conventionally include a promoter operably linked to the polynucleotide, a transcription termination signal, a ribosome-binding domain, and a translation termination signal.

The method of transforming the vector of the present invention includes any method of introducing a nucleic acid into a cell, and can be performed by selecting an appropriate standard technique as known in the art depending on the host cell. For example, the transformation may be carried out via particle bombardment, electroporation, calcium phosphate (CaPO₄) precipitation, calcium chloride (CaCl₂) precipitation, microinjection, a polyethylene glycol (PEG) technique, a DEAE-dextran technique, a cationic liposome technique, a lithium acetate-DMSO technique, but the method is not limited thereto.

As used herein, the term “host cell” refers to a eukaryotic cell into which a nucleic acid molecule having the activity of the protein secretory factor of the present invention is introduced and can act as a signal peptide. The host cell may include, for example, generally known eukaryotic hosts such as yeasts; insect cells such as Spodoptera frupperda; and animal cells such as CHO, COS1, COS7, BSC1, BSC40, and BMT10, but is not limited thereto.

In the present invention, examples of the host cell may be an animal host cell, and specifically, it may be a Chinese hamster ovary cell (CHO cell), but is not limited thereto.

In one specific embodiment of the present invention, the Chinese Hamster Ovary (CHO) cell, which is widely used in the production of recombinant proteins was used as the host cell (Example 4).

As used herein, the term “transformant” refers to a transformed animal cell in which an expression vector including a signal peptide consisting of an amino acid sequence of SEQ ID NO: 1, SEQ ID NO: 2, SEQ ID NO: 3, SEQ ID NO: 4, or SEQ ID NO: 5 and a target protein is introduced into a CHO cell, which is the host cell.

In one specific embodiment of the present invention, it was confirmed that the transformant increased the expression levels of the light and heavy chains of pembrolizumab (i.e., an antibody) which is the target protein (Example 4).

Still another aspect of the present invention provides a method for producing a target protein, including:
i) culturing a transformed cell including an expression vector for secreting a target protein, which includes an expression cassette in which a nucleic acid sequence encoding a protein secretory factor consisting of an amino acid sequence of SEQ ID NO: 1, SEQ ID NO: 2, SEQ ID NO: 3, SEQ ID NO: 4, or SEQ ID NO: 5; and a gene encoding a target protein are operably linked; and
ii) recovering the target protein from the culture medium or culture supernatant of the cultured cells.

The “protein secretory factor”, “target protein”, “operably linked”, “expression cassette”, “expression vector for secreting a target protein”, “host cell”, and “transformant” of the present invention are as described above.

As used herein, the term “culturing” refers to a process of growing the transformed cells in appropriately, artificially controlled environmental conditions. In the present invention, the method for producing a target protein using the CHO cells as the host cell may be performed using a method widely known in the art. Specifically, the culturing may be performed by a batch process, a fed batch or repeated fed batch process in a continuous manner, but is not limited thereto.

The medium used for the culturing should satisfy the requirements for a specific strain in an appropriate manner. Carbon sources that may be used in the present invention may include sugars and carbohydrates such as glucose, sucrose, lactose, fructose, maltose, starch, and cellulose; oils and fats such as soybean oil, sunflower oil, castor oil, and coconut oil; fatty acids such as palmitic acid, stearic acid, and linoleic acid; alcohols such as ethanol; and organic acids such as gluconic acid, acetic acid, and pyruvic acid, but these are not limited thereto. These substances may be used alone or in a mixture.

Nitrogen sources that may be used in the present invention may include peptone, yeast extract, meat extract, malt extract, corn steep liquor, defatted soybean cake, and urea or inorganic compounds, for example, ammonium sulfate, ammonium chloride, ammonium phosphate, ammonium carbonate, and ammonium nitrate, but these are not limited thereto. These nitrogen sources may also be used alone or in a mixture.

Phosphorus sources that may be used in the present invention may include potassium dihydrogen phosphate or dipotassium hydrogen phosphate, or corresponding sodium-containing salts, but these are not limited thereto. In addition, the culture medium may contain a metal salt such as magnesium sulfate or iron sulfate, which is required for the growth. Lastly, in addition to the above-described substances, essential growth substances such as amino acids and vitamins may be used. Additionally, suitable precursors may be used in the culture medium. These substances may be appropriately added to the medium during culturing in a batch or continuous manner.

Basic compounds such as sodium hydroxide, potassium hydroxide, or ammonia, or acidic compounds such as phosphoric acid or sulfuric acid may be added to the culture medium in a suitable manner to adjust the pH of the culture medium. In addition, an anti-foaming agent such as fatty acid polyglycol ester may be used to suppress the formation of bubbles. In order to maintain the culture medium in an aerobic state, oxygen or oxygen-containing gas may be injected into the culture medium. The temperature of the culture medium may be usually 20° C. to 45° C., preferably 25° C. to 40° C., but may be changed depending on conditions and is not limited thereto.

In one specific embodiment of the present invention, the recombinant expression vectors (i.e., pCB-SP7.2-Pem, pCB-Clus-Pem, pCB-Pig-Pem, and pCB-CC-Pem) were introduced into the host CHO cells (ExpiCHO-S™ cells) and cultured in 30 mL of an ExpiCHO expression medium (CHO expression medium) for 12 days via a fed-batch culture (Example 4).

The method of the present invention for producing a target protein may include a step of recovering the target protein from the culture medium. As used herein, the term “recovery” is a process of obtaining the target protein from the culture medium, and may be performed using methods known in the art, for example, centrifugation, filtration, anion-exchange chromatography, crystallization, HPLC, etc., but the method is not limited thereto.

The recovery step may include a purification process, and those skilled in the art may select and utilize among various known purification processes as needed. For example, the host cell can be separated from the culture medium or culture supernatant of the host cell by the conventional chromatographic methods such as immunoaffinity chromatography, receptor affinity chromatography, hydrophobic interaction chromatography, lectin affinity chromatography, size-exclusion chromatography, cation or anion exchange chromatography, high performance liquid chromatography (HPLC) and reversed-phase HPLC. In addition, when the desired protein is a fusion protein with a specific tag, label, or chelate moiety, it can be purified by a specific binding partner or drug. The purified protein may be cleaved into a desired protein region, such as the removal of the secretory factor, or it can remain as it is. A desired form of protein including additional amino acid may be produced by cutting the fusion protein during the cutting process.

The protein secretory factor consisting of the amino acid sequence of SEQ ID NO: 1, SEQ ID NO: 2, SEQ ID NO: 3, SEQ ID NO: 4, or SEQ ID NO: 5 of the present invention may be a secretory factor that is accurately cleaved at the N-terminal cleavage site of the target protein.

The signal peptide is located at the N-terminal region of the recombinant protein to be produced, and when the target protein is translocated, it is degraded by a signal peptidase. However, existing protein secretory factors can often degrade the quality of target proteins due to the mis-cleavage problem. The “mis-cleavage” refers to a phenomenon where the signal peptide is not completely degraded at the correct position and the signal peptide sequence partially remains at the N-terminus of the target protein.

In one specific embodiment of the present invention, the cleavage of the protein secretory factors (signal peptides) was confirmed using the purified target proteins by a Q-TOF MS mass spectrometer. As a result, it was confirmed that 100% cleavage was observed at the predicted cleavage sites.

Accordingly, the expression vector including the protein secretory factor (signal peptide) of the present invention increases the productivity of the target protein through efficient expression and secretion of the recombinant protein, and can be a powerful genetic tool to solve the mis-cleavage problem.

### Advantageous Effects

The protein secretory factor of the present invention, that is, the signal peptide can significantly increase the productivity of recombinant proteins through high-level expression and can be expected to be used as a powerful genetic tool, which can solve the mis-cleavage problem of the conventional signal peptides by 100% cleavage at cleavage sites.

### BRIEF DESCRIPTION OF DRAWINGS

FIGS. 1(a) to 1(j) are graphs is a graph predicting the signal peptides using SignalP4.1.

FIG. 2 is a diagram confirming the expression levels of signal peptide-mCherry through temporary expression.

FIG. 3 is a diagram showing a vector map for site specific integration.

FIG. 4 is a diagram comparing the expression levels of mCherry in the site-specific integrated cells.

FIGS. 5(a) to 5(c) are graphs is a graph showing mass data that confirms the cleavage of an anti-PD-1 antibody fused with SP7.2 and Clus.

### DETAILED DESCRIPTION OF PREFERRED EMBODIMENTS

Hereinafter, the present invention will be described in more detail by way of Examples. However, these Examples are given for illustrative purposes only, and the scope of the invention is not intended to be limited to or by these Examples

### Example 1. Preparation of Novel Signal Peptide Sequences Derived from CHO Cells

### 1-1. CHO HCP Mass Analysis

Four types (ADH, BSA, PHO, and ENL) of MassPREPTM Protein Digest Standard were added to the culture medium of CHO cells (DXB11) treated with trypsin as follows. Among the four types of MassPREPTM Protein Digest Standard, PHO was used as an internal standard for calculating the concentration of each host cell protein (HCP). Each sample was analyzed for host cell protein (HCP) using a 2D LC (high pH RP/Low pH RP)-Q-TOF (UDMSe) method.

MS data (UDMSe) was obtained for each fraction by injecting directly into Q-TOF MS in the 2D column. The MS data (UDMSe) of the 10 fractions obtained in the above manner were merged into one data using ProteinLynx Global SERVER (PLGS, Ver. 3.0.2) Software. Thereafter, the HCP was identified using the merged data of each sample and the Chinese Hamster Protein Database, and the concentration of each HCP was calculated using PHO as an internal standard.

### 1-2. Selection of Signal Peptides From CHO HCP Data

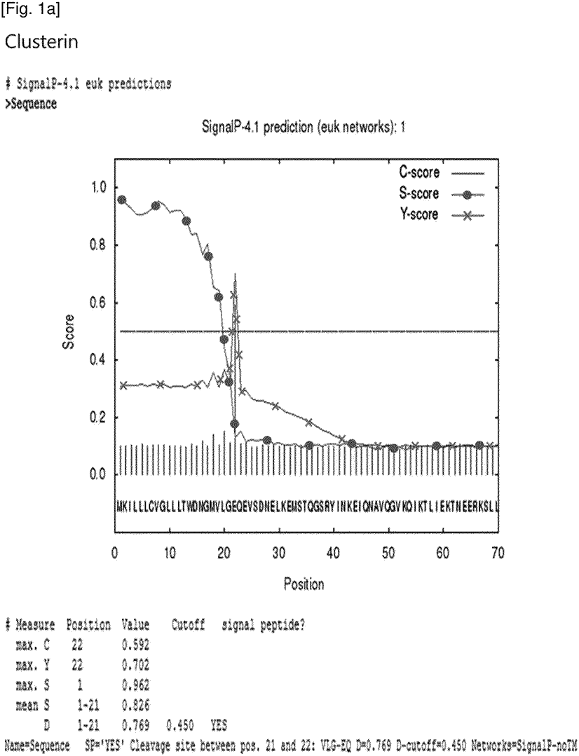

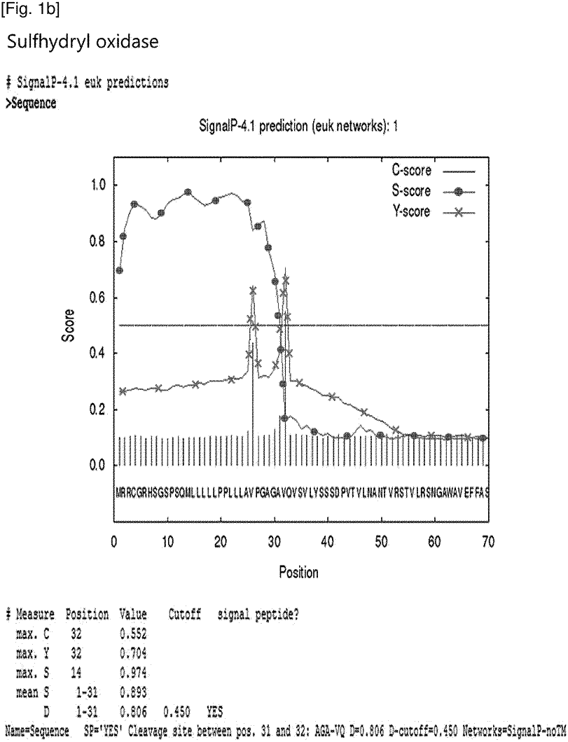

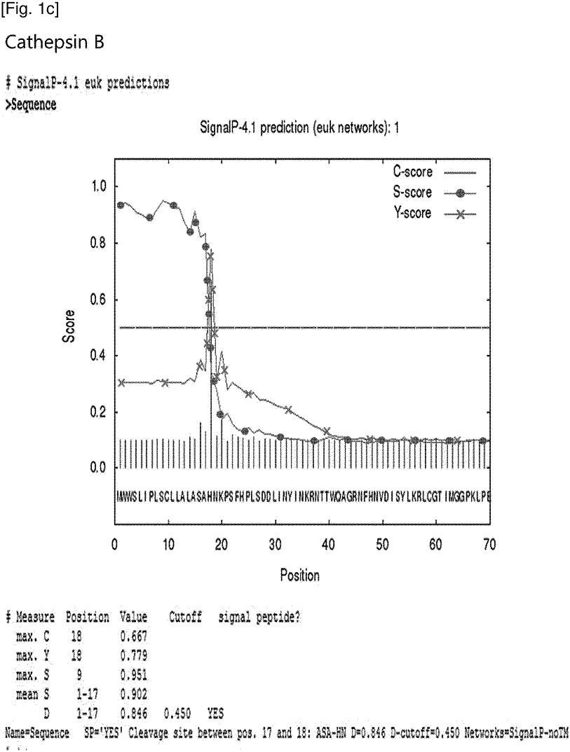

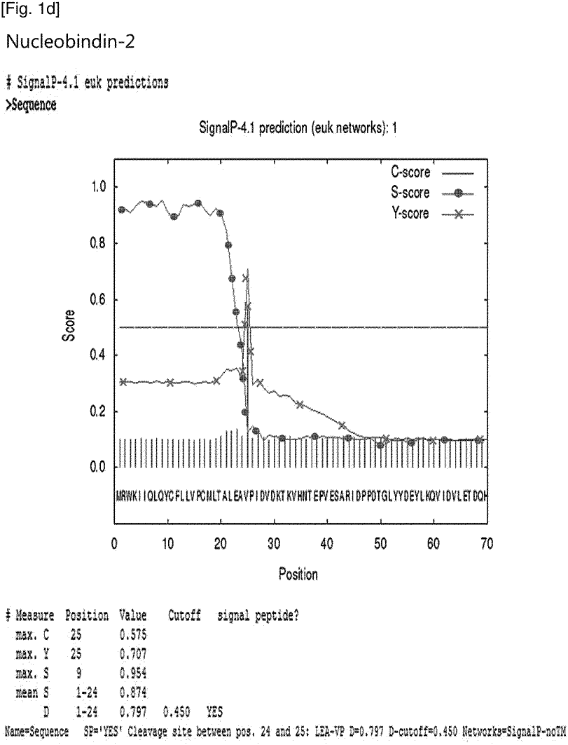

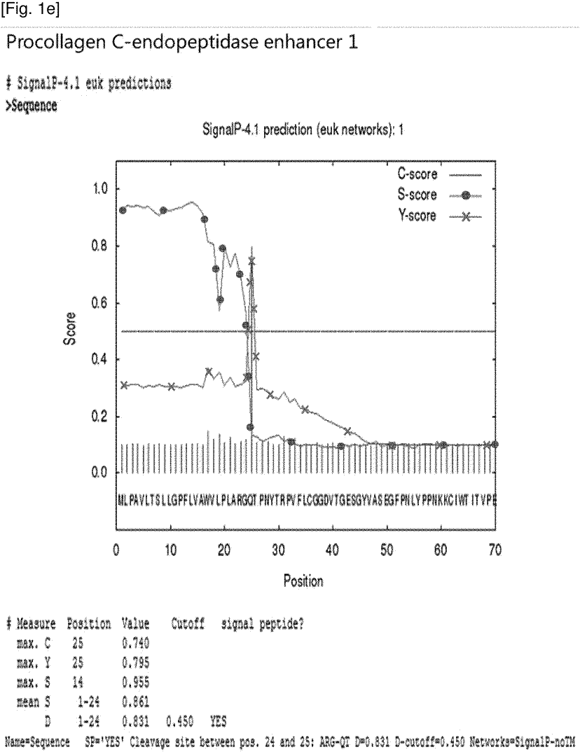

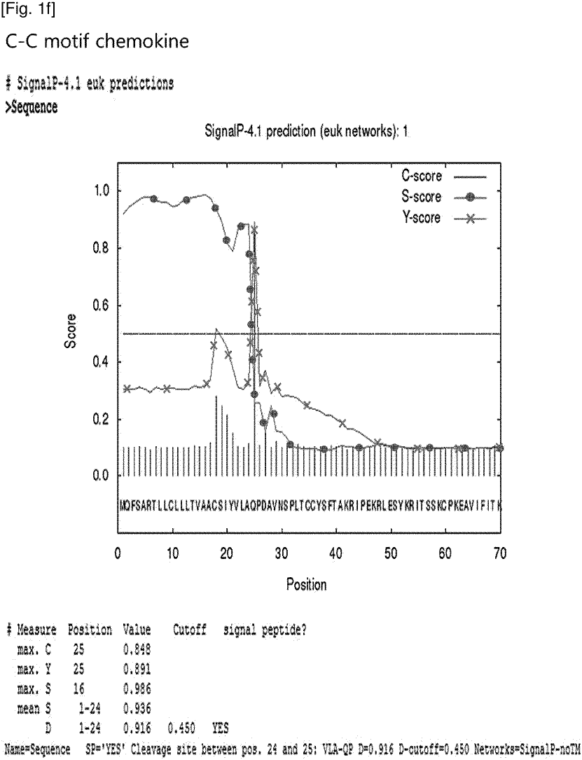

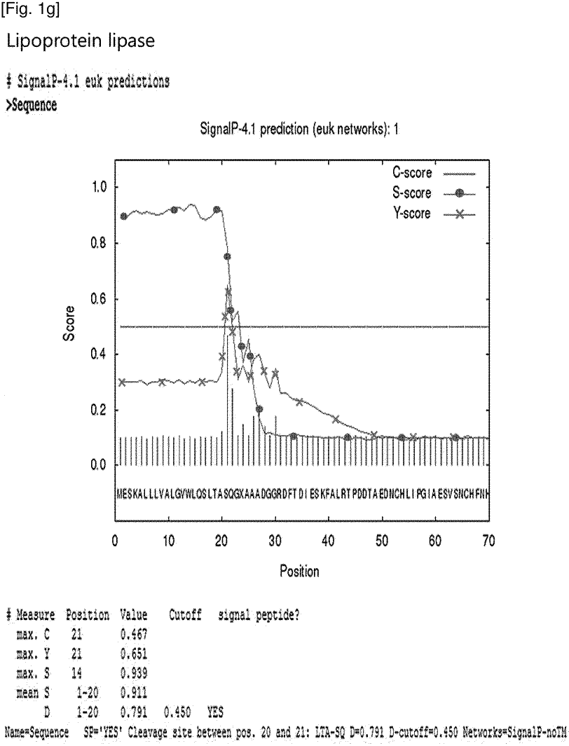

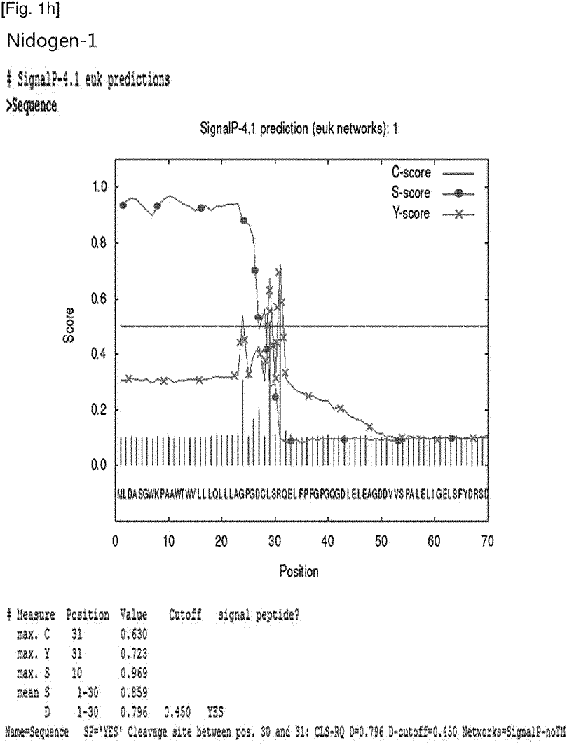

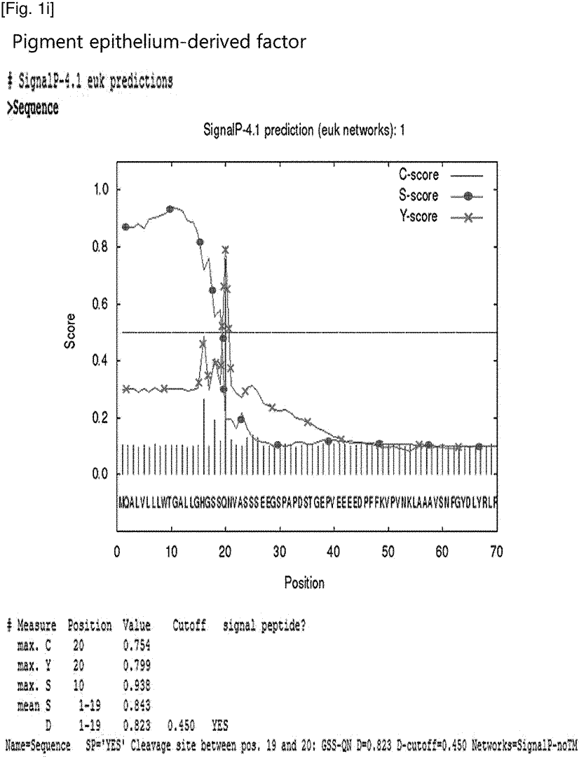

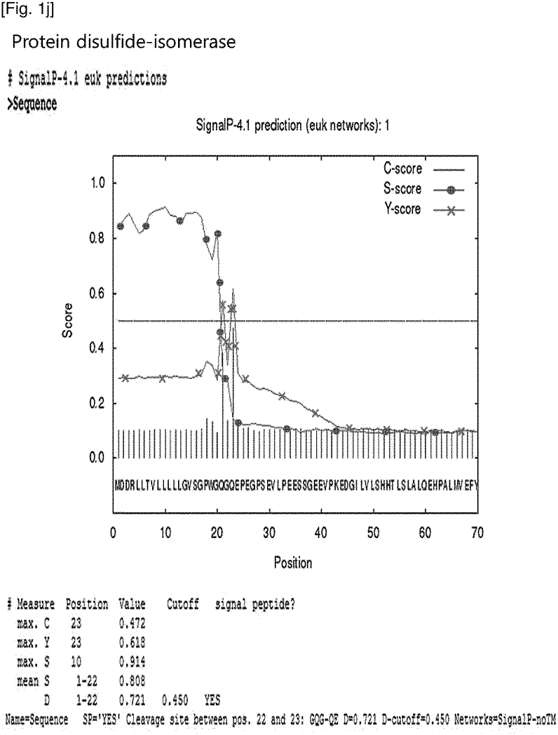

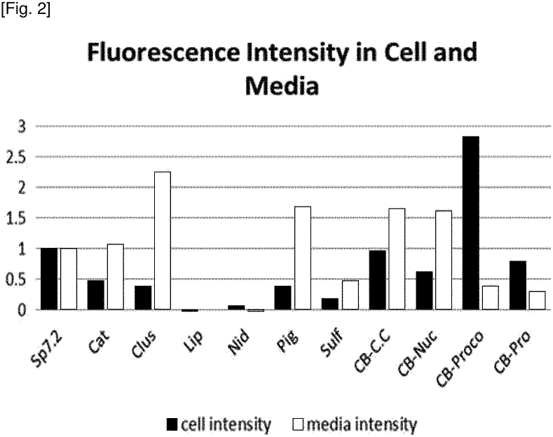

The proteins were arranged in the order of high concentrations, and the amino acid sequence of each protein was confirmed from the CHO Genome Database (http://chogenome.org). The thus-obtained amino acid sequences were entered into SignalP4.1 Server (http://www.cbs.dtu.dk/services/SignalP/) to predict the presence of secretory proteins and signal peptide sequences (Table 1, FIGS. 1 and Table 4).

TABLE 1

HCP Secretion Protein Predicted via SignalP4.1

| Protein | SEQ ID NO: | SP (Signal Peptide) Sequence | SEQ ID NO: | DNA Sequence |
|---|---|---|---|---|
| Cathepsin B (Cat) | 1 | MWWSLIPLSC LLALASA | 11 | ATG TGG TGG TCC TTG ATT CCG CTC TCT TGC CTG CTG GCA CTG GCA AGT GCC |
| C—C motif chemokine | 2 | MQFSARTLLC LLLTVAACSI YVLA | 12 | ATG CAG TTC TCC GCA AGA ACG CTT CTG TGC CTG CTA CTC ACA GTT GCT GCC TGT AGC ATC TAT GTG CTG GCC |
| Nucleobindin-2 (Nuc) | 3 | MRWKIIQLQY CFLLVPCMLT ALEA | 13 | ATG AGG TGG AAG ATC ATC CAG CTA CAG TAC TGT TTT CTT TTG GTC CCG TGC ATG CTT ACT GCT CTG GAA GCT |
| Clusterin (Clus) | 4 | MKILLLCVGL LLTWDNGMVL G | 14 | ATG AAG ATT CTC CTG TTG TGC GTG GGG CTG CTG CTG ACC TGG GAC AAT GGC ATG GTC CTG GGA |
| Pigment epithelium-derived factor (Pig) | 5 | MQALVLLLWT GALLGHGSS | 15 | ATG CAG GCC CTG GTG CTA CTC CTC TGG ACA GGA GCC CTG CTT GGG CAT GGC AGC AGC |
| Procollagen C-endopeptida se enhancer 1 (Proco) | 6 | MLPAVLTSLL GPFLVAWVLP LARG | 16 | ATG CTG CCT GCT GTC CTA ACC TCC CTC CTG GGG CCA TTC CTT GTG GCC TGG GTA CTG CCT CTT GCC CGA GGC |
| Sulfhydryl oxidase (Sulf) | 7 | MRRCGRHSGS PSQMLLLLLP PLLLAVPGAG A | 17 | ATG AGG AGG TGC GGC CGC CAC TCG GGG TCG CCG TCG CAG ATG CTA CTG CTG CTG CTG CCG CCG CTG CTG CTC GCG GTG CCC GGC GCT GGC GCG |
| Lipoprotein lipase (Lip) | 8 | MESKALLLVA LGVWLQSLTA | 18 | ATG GAG AGC AAA GCC CTG CTC CTG GTG GCT CTG GGA GTG TGG CTC CAG AGT TTG ACC GCC |
| Nidogen-1 (Nid) | 9 | MLDASGWKPA AWTWVLLLQL LLAGPGDCLS | 19 | ATG CTG GAC GCG AGC GGC TGG AAG CCC GCG GCG TGG ACA TGG GTG CTG CTG CTG CAG CTA TTG CTG GCG GGG CCC GGA GAC TGC CTG AGC |
| Protein disulfide-isom erase (Pro) | 10 | MDDRLLTVLL LLLGVSGPWG QG | 20 | ATG GAT GAT CGG CTC CTG ACA GTG TTG CTG CTC CTG CTG GGT GTC TCA GGC CCA TGG GGA CAG GGA |

### Example 2. Selection of CHO-Derived High Efficiency Signal Peptides via Temporary Expression

### 2-1. Preparation of Recombinant Protein Expression Vectors for Temporary Expression

In order to confirm whether the 10 types of signal peptides selected in Example 1 can be used as general secretory factors, mCherry (pmCherry Vector, Clontech, 632522) protein was selected as the target protein.

The sequence of the polynucleotide of the gene encoding the mCherry protein is shown in Table 2.

TABLE 2

| Protein | SEQ ID NO: | Amino Acid Sequence | SEQ ID NO: | DNA Sequence |
|---|---|---|---|---|
| mCherry | 31 | MVSKGEEDNMAIIKEFMRFKVHMEGSVNGHEFEIEGEGEGRPYEGTQTAKLKVTKGGPLPFAWDILSPQMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNKLDITSHNEDYTIVEQYERAEGRHSTGGMDELYK | 32 | ATGGTGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAGTTCATCTACAAGGTGAAGCTGCGCGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTGAGGTCAAGACCACCTACAAGGCCAAGAAGCCCGTGCAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGTAA |

Based on the gene encoding the mCherry protein, PCR was performed using the signal peptide sequences identified from the CHO HCP Mass Data and primers containing Kpnl/Xhol, and the mCherry expressed by the 10 types of signal peptide sequences was constructed.

When the length of the signal peptide was long, the primer was divided into two and PCR was performed twice. The mCherry PCR products containing the 10 types of signal peptide sequences were cleaved with Kpnl and Xhol and then cloned into pcDNA3.1 (+) (Invitrogen, Cat. No. V790-20) to construct expression vectors.

Additionally, for use as a positive control, a mCherry protein expression vector fused with the SP7.2 signal peptide (Korean Patent Laid-Open Publication 10-2015-0125402 A), which is a known secretory factor, was prepared in the same manner. The sequence of the SP7.2 signal peptide is shown in Table 3.

TABLE 3

| Protein | SEQ ID NO: | SP Sequence | SEQ ID NO: | DNA Sequence |
|---|---|---|---|---|
| SP7.2 | 33 | MHRPEAMLLLLTLALLGGPTWA | 34 | ATGCACCGGCCAGAGGCCATGCTGCTGCTGCTCACGCTTGCCCTCCTGGGGGGCCCCACCTGGGCA |

TABLE 4

| | | | | |
|---|---|---|---|---|
| Protein | GeneBank No. | Amino Acid Sequence | Expected SP Sequence from SignalP4.1 | DNA Sequence |
| Clusterin | XP_007643887.1 | 1 MKILLLCVGL LLTWDNGMVL GEQEVSDNEL KEMSTQGSRY INKEIQNAVQ GVKQIKTLIE 61 KTNEERKSLL NSLEEAKKKK EDALDDTRDT EMKLKAFPEV CNETMMALWE ECKPCLKQTC 121 MKFYARVCRS GSGLVGRQLE EFLNQSSPFY FWMNGDRIDS LMESDRQQSQ VLDAMQDSFT 181 RASGIMDMLF QDRFFTHEPQ DTHYFSPFGF PHRRPHFLYP KSRLVRSLIP LSHYGPPSFH 241 DMFQPFLEMI HQAQQAMDVQ FHRPAFQFPD KGLREGEDDR AVCKEIRHNS TGCLKMKGQC 301 EKCQEILSVD CSANNPAQAH LRQELNDSLQ MAERLTQQYN ELLHSLQTKM LNTSSLLEQL 361 NEQFNWVSQL ANLTQGEDQY YLRVSTVTTH SNNSEEPSRV TEVWKLFDS DPITWLPEE 421 VSKDNPKFMD TVAEKALQEY RKKSRAE | MKILLLCVGL LLTWDNGMVL G | ATG AAG ATT CTC CTG TTG TGC GTG GGG CTG CTG CTG ACC TGG GAC AAT GGC ATG GTC CTG GGA |
| Sulfhydryl oxidase | XP_007639037.1 | 1 MRRCGRHSGS PSQMLLLLLP PLLLAVPGAG AVQVSVLYSS SDPVTVLNAN TVRSTVLRSN 61 GAWAVEFFAS WCGHCIAFAP TWKELAYDVR EWRPVLNLAV LDCAEETNTA VCRDFNISGF 121 PTVRFFKAFS KNGSGITLPV ADASVETLRR KLIDALESHS DMWSSSRPKL KPAKLVEINE 181 FFAETNEDYL VLIFEDKDSY VGREVTLDLF QHHIPVHRVL NTERNAVSKF GWEFPSCYL 241 LFRNGSFSRV PWMESRLFY TSYLKGMSGP ILVDPPTTTI STDAPVTTDV VPTVWKVANH 301 ARIYMADLES SLHYIFLVEV GKFSVLEGQR LLALKKLVAV LAKYFPGRPL AQNFLHSIHD 361 WLQRQQRKKI PYKFFRAALD NRKEGIVLTE KVNWVGCQGS KPHFRGFPCS LWILFHFLTV 421 QASRYSENHP QEPADGQEVL QAMRSYVQWF FGCRDCAEHF ENMAASTMHR VRSPTSAVLW 481 LWTSHNKVNA RLSGAPSEDP YFPKVQWPLR ELCFDCHNEI NGREPVWDLE ATYRFLKAHF 541 SSENIILDTP VAGLATQRNP QILGATPEPH M | MRRCGRHSGS PSQMLLLLLP PLLLAVPGAG A | ATG AGG AGG TGC GGC CGC CAC TCG GGG TCG CCG TCG CAG ATG CTA CTG CTG CTG CTG CCG CCG CTG CTG CTC GCG GTG CCC GGC GCT GGC GCG |
| Cathepsin B | XP_003498026.1 | 1 MWWSLIPLSC LLALASAHNK PSFHPLSDDL INYINKRNTT WQAGRNFHNV DISYLKRLCG 61 TIMGGPKLPE RVAFAEDMEL PENFDAREQW SNCPTIKQIR DQGSCGSCWA FGAVGAMSDR 121 LCIHTNGHVN VEVSAEDLLT CCGSQCGDGC NGGYPSGAWN FWIKKGLVSG GLYNSHVGCL 181 PYTIPPCEHH VNGSRPQCTG EGDTPKCTKS CEAGYSPSYK EDKHYGYTSY SVSNNEKEIM 241 AEIYKNGPVE GAFTVFSDFL TYKSGVYKHE AGDIMGGHAI RILGWGVENS VPYWLVANSW 301 NVDWGDNGLF KILRGEDHCG IESEIVAGIP RTDLYWGRF | MWWSLIPLSC LLALASA | ATG TGG TGG TCC TTG ATT CCG CTC TCT TGC CTG CTG GCA CTG GCA AGT GCC |
| Nucleobindin-2 | XP_003513452.1 | 1 MRWKIIQLQY CFLLVPCMLT ALEAVPIDVD KTKVHNTEPV ESARIDPPDT GLYYDEYLKQ 61 VIDVLETDQH FREKLQKADI EEIRSGRLSK ELDLVSHHVR TKLDELKRQE VARLRMLIKA 121 KLDSLQDTGM NHHLLLKQFE HLNHQNPDTF ESSDLDMLIK AATADLEQYD RTRHEEFKKY 181 EMMKEHERRE YLKTLNEEKR KEEESKFEEM KRKHENHPKV NHPGSKDQLK EVWEEADGLD 241 PNDFDPKTFF KLHDVNNDGF LDEQELEALF TRELEKVYDP RNAEDDMIEM EEERLRMREH 301 VMNEIDNNKD RLVTLEEFLR ATEKKEFLEP DSWETLGQQQ LFTEEELKEY ESIIAMQENE 361 LKKRADELQK QKEELQRQHD HLEAQKQEYQ QAVQQLEHKK FQQGIAPSGP AGELEFKPRM | MRWKIIQLQY CFLLVPCMLT ALEA | ATG AGG TGG AAG ATC ATC CAG CTA CAG TAC TGT TTT CTT TTG GTC CCG TGC ATG CTT ACT GCT CTG GAA GCT |
| Procollagen C-endopeptidase enhancer 1 | XP_003510679.1 | 1 MLPAVLTSLL GPFLVAWVLP LARGQTPNYT RPVFLCGGDV TGESGYVASE GFPNLYPPNK 61 KCIWTITVPE GQTVSLSFRV FDMELHPSCR YDALEVFAGS GTSGQRLGRF CGTFRPAPVV 121 APGNQVTLRM TTDEGTGGRG FLLWYSGRAT SGTEHQFCGG RMEKAQGTLT TPNWPESDYP 181 PGISCSWHII APSDQVIMLT FGKFDVEPDT YCRYDSVSVF NGAVSDDSKR LGKFCGDKAP 241 SPISSEGNEL LVQFVSDLSV TADGFSASYK TLPRDAVEKE LAPSPGEDVQ LGPQSRSDPK 301 TGTGPKVKPP SKPKFQPAEK PEVSPDTQET PVAPDPPSAT CPKQYKRLGT LQSNFCASSL 361 VVTGTVKTMV RGPGEGLTVT ISLLGVYKSG GLDLPSPPTD TSLKLYVPCR QMPPMKKGAS 421 YLLMGQVEEN RGPILPPESF LVPYKPNQDQ ILNNLRKRKC PSQPRPAA | MLPAVLTSLL GPFLVAWVLP LARG | ATG CTG CCT GCT GTC CTA ACC TCC CTC CTG GGG CCA TTC CTT GTG GCC TGG GTA CTG CCT CTT GCC CGA GGC |
| C—C motif chemokine | XP_003495840.1 | 1 MQFSARTLLC LLLTVAACSI YVLAQPDAVN SPLTCCYSFT AKRIPEKRLE SYKRITSSKC 61 PKEAVIFITK LKREICADPK QDWVQTYTKK LDQSQAKSEA ATVYKTAPLN ANLTHESAVN 121 ASTTAFPTTD LRTSVRVTSM TVN | MQFSARTLLC LLLTVAACSI YVLA | ATG CAG TTC TCC GCA AGA ACG CTT CTG TGC CTG CTA CTC ACA GTT GCT GCC TGT AGC ATC TAT GTG CTG GCC |
| Lipoprotein lipase | XP_003499976.1 | 1 MESKALLLVA LGVWLQSLTA SQGXAAADGG RDFTDIESKF ALRTPDDTAE DNCHLIPGIA 61 ESVSNCHFNH SSKTFVVIHG WTVTGMYESW VPKLVAALYK REPDSNVIW DWLYRAQQHY 121 PVSAGYTKLV GNDVARFINW MEEEFNYPLD NVHLLGYSLG AHAAGVAGSL TNKKVNRITG 181 LDPAGPNFEY AEAPSRLSPD DADFVDVLHT FTRGSPGRSI GIQKPVGHVD IYPNGGTFQP 241 GCNIGEAIRV IAERGLGDVD QLVKCSHERS IHLFIDSLLN EENPSKAYRC NSKEAFEKGL 301 CLSCRKNRCN NVGYEINKVR AKRSSKMYLK TRSQMPYKVF HYQVKIHFSG TESDKQLNQA 361 FEISLYGTVA ESENIPFTLP EVSTNKTYSF LIYTEVDIGE LLMMKLKWKS DSYFSWSDWW 421 SSPGFVIEKI RVKAGETQKK VIFCAREKVS HLQKGKDSAV FVKCHDKSLK KSG | MESKALLLVA LGVWLQSLTA | ATG GAG AGC AAA GCC CTG CTC CTG GTG GCT CTG GGA GTG TGG CTC CAG AGT TTG ACC GCC |
| Nidogen-1 | XP_003507635.2 | 1 MLDASGWKPA AWTWVLLLQL LLAGPGDCLS RQELFPFGPG QGDLELEAGD DWSPALELI 61 GELSFYDRSD ITSVYVTTNG IIAMSEPPAR ESHPGTFPPS FGSVAPFLAD LDTTDGLGNV 121 YYREDLSPSI MQMAAEYVQR GFPEVPFQPT SWWTWESV APYEGPSGSS AQEGKRNTFQ 181 AVLASSNSSS YAIFLYPEDG LQFFTTFSKK DENQVPAMVG FSQGLVGFLW RSDGAYNIFA 241 NDRESIENLA KSSNAGHQGV WVFEIGSPAT AKGVVSADVN LGLDDDGSDY EDEEYDLATS 301 HLGLEDMATQ PFPSPSPRRG NTHPHDVPRV LSPSYEATER PHGIPTERTR TFQLPAERFH 361 QQHPQVIDVD EVEETGIVFS YNIGSQQTCA NNRHQCSVHA ECRDYATGFC CRCVANYTGN 421 GRQCVAEGSP QRVNGKVKGR IFVGNSQVPV VFENTDLHSY VVMNHGRSYT AISTIPETVG 481 YSLLPLAPIG GIIGWMFAVE QNGFKNGFSI TGGEFTRQAE VTFLGHPGKL VLKQHFSGID 541 EHGHLTINTE LDGRVPQIPY GSSVHIEPYT ELYHYSSSVI TSSSTREYTV TEPDPDGTAP 601 SHTHVYQWRQ TITFQECVHD DSRPALPSTQ QLSVDSVFVL YNQEERILRY ALSNSIGPVR 661 EGSPDALQNP CYIGTHGCDS NAACRPGPGT QFTCECSIGF RGDGQTCYDI DECSEQPSRC 721 GNHAACNNSP GAYLCECVEG YHFSDGGICV ADVDQRPINY CETGLHNCDI PQRAQCIYMG 781 GSSYTCSCLP GFSGDGRACQ DVDECQLSRC HPDAFCYNTP GSFTCQCKPG YQGDGFQCVP 841 GEVGKTRCQL EREHILGASG VADAQQPRLL GMYVPQCDEY GHYEPTQCHH GTGYCWCVDR 901 DGRELEGTRT QPGMRPPCLS TVAPPIHQRP VVPTAVIPLP PGTHLLFAQT GKIERLPLEG 961 NTMKKTEAKA FFHIPAKVII GLAFDCVDKV VYWTDISEPS IGRASLHGGE PTTIIRQDLG 1021 SPEGIALDHLGRNIFWTDSQ LDRIEVARMD GTQRRVLFDT GLVNPRGIVT DSVGGNLYWT 1081 DWNRENPKIE TSYMDGTNRR ILAQDNLGLP NGLTFDAFSS QLCWVDAGTH RAECLNPAQP 1141 SSRKVLEGLQ YPFAVTSYGK NLYYTDWKTN SVIAMDLAIS KEMDAFTPTS RPGYMASPLP 1201 CPNALKATTT AQ | MLDASGWKPA AWTWVLLLQL LLAGPGDCLS | ATG CTG GAC GCG AGC GGC TGG AAG GCG GCG GCG TGG ACA TGG GTG CTG CTG CTG CAG CTA TTG CTG GCG GGG CCC GGA GAC TGC CTG AGC |
| Pigment epithelium-derived factor | XP_003515170.1 | 1 MQALVLLLWT GALLGHGSSQ NVASSSEEGS PAPDSTGEPV EEEEDPFFKV PVNKLAAAVS 61 NFGYDLYRLR SSASPTANVL LSPLSVATAL SALSLGAEQR TESIIHRALY YDLISNSDIH 121 STYKELLASV TAPEKSLKSA SRIVFERKLR VRSSFVAPLE KSYGTRPRIL TGNPRIDLQE 181 INNWIQAQMK GKLARSTREM PSAISILLLG VAYFKGQWVT KFDSRKTSLQ DFHLDEDRTV 241 KVPMMSEPKA ILRYGLDSDL NCKVWEHGGW EGSERGRVSS IRKSIWGYSK IHELQSLFES 301 PDFSKITGKP VKLTQVEHRA AFEWNEEGVE TSPNPGLQPV RLTFPLDYHL NQPFIFVLRD 361 TDTGALLFIG KILDPRGT | MQALVLLLWT GALLGHGSS | ATG CAG GCC CTG GTG CTA CTG CTG TGG ACA GGA GCC CTG CTT GGG CAT GGC AGC AGC |
| Protein disulfide-isomerase | XP_003501525.1 | 1 MDDRLLTBVLL LLLGVSGPWG QGQEPEGPSE VLPEESSGEE VPKEDGILVL SHHTLSLALQ 61 EHPALMVEFY APWCGHCKAL APEYSKAAAL LAAESASVTL AKVDGPAEPE LTKEFGWGY 121 PTLKFFQNGN RTNPEEYTGP QKAEGIAEWL RRRVGPSAKR LEDEEDVQAL TDKWEWVIG 181 FFQDLQGEDV ATFLALARDA LDITFGFTDQ PQLFQKFGLT KDTVILFKKF DEGRADFPVD 241 KDTGLDLGDL SRFLVTHSMH LVTEFNSQTS PKIFAAKILN HLLLFVNKTL AQHRELLTDF 301 REAAPPFRGQ VLFVMVDVAA DNDHVLNYFG LKAEEAPTLR LINVETTKKY APTGLVPITA 361 ASVAAFCQAV LHGQVKPYLL SQEIPPDWDE RPVKTLVGKN FEQVAFDETK NVFVKFYAPW 421 CSHCKEMAPA WEALAEKYRD REDIVIAELD ATANELEAFS VHGYPTLKFF PAGPDRKVIE 481 YKSTRDLETF SKFLDSGGNL PEEEPKEPAI STPEIQDNST VGPKEEL | MDDRLLTVLL LLLGVSGPWG QG | ATG GAT GAT CGG CTC CTG ACA GTG TTG CTG CTC CTG CTG GGT GTC TCA GGC CCA TGG GGA CAG GGA |

### 2-2. Temporary Expression

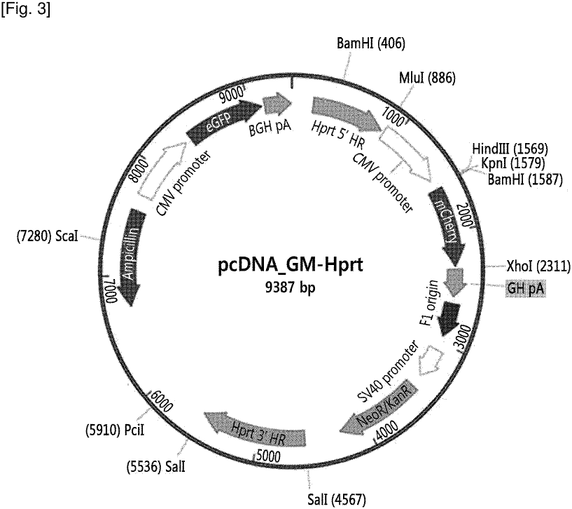

Each of the mCherry expression vectors expressed by the 10 types of signal peptides were transfected (1 mL) according to the CHO—S cell line Amaxa 4D-Nucleofector protocol. Thereafter, on the 2^{nd} and 6^{th} days, the fluorescence values of the intracellular fluorescence and the fluorescent proteins secreted from the culture medium were measured (FIG. 3).

In the case of the intracellular fluorescence, FACS (Accuri) was used to measure the average value in the histogram of the portion higher than that of the negative control (empty vector, pMaxGFP).

In the case of the fluorescence values of the fluorescent proteins secreted into the culture medium, 100 µ0 was sampled on the 2^{nd} and 6^{th} days, and then centrifuged to obtain the supernatant only, and fluorescence was measured at 587/610 nm using a multiple reader.

It was confirmed that the signal peptides with a high fluorescence value measured in the culture medium were Cat, CC, Nuc, Clus, and Pig. Additionally, the four types of secretory factors (Clus, Pig, Nuc, and CC) which showed the expression higher than the positive control SP7.2, SP7.2 to be used as the positive control, and one type of signal peptide with a high fluorescence value in cells (Proco) were selected as a negative control.

### Example 3. Comparison of Expression Through Site-Specific Integration

### 3-1. Construction of Expression Vectors for Site-Specific Integration

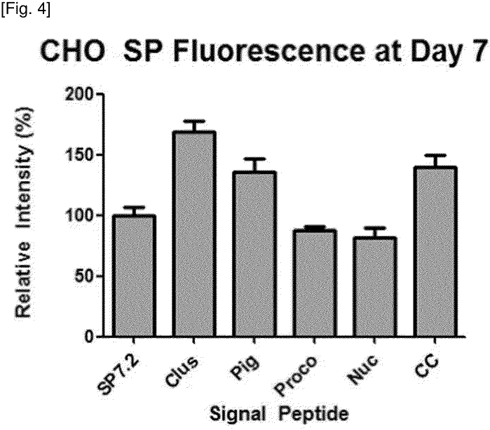

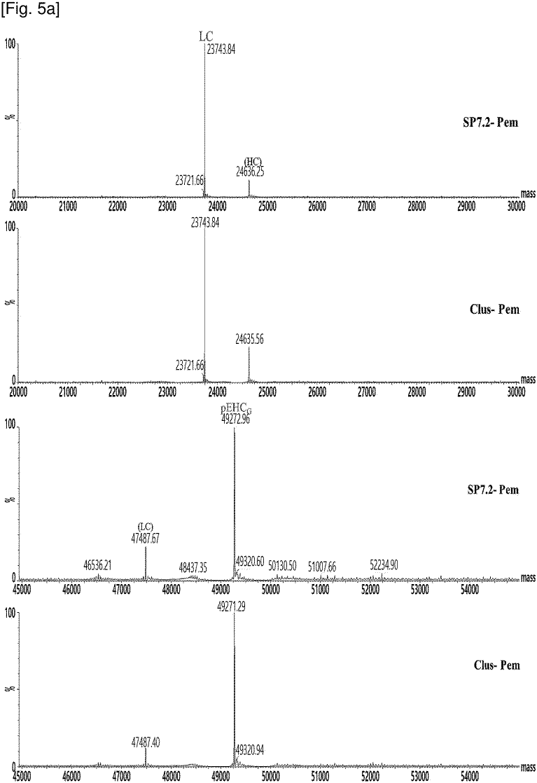

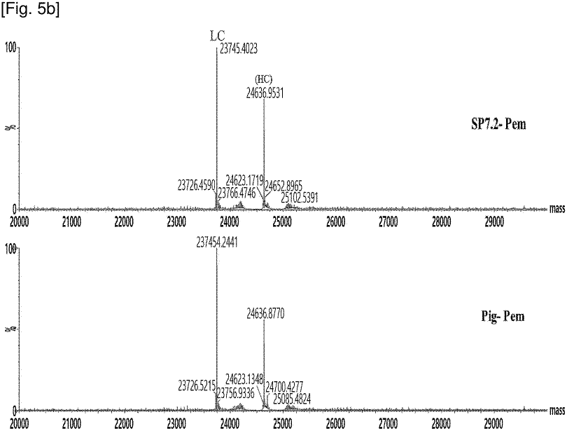

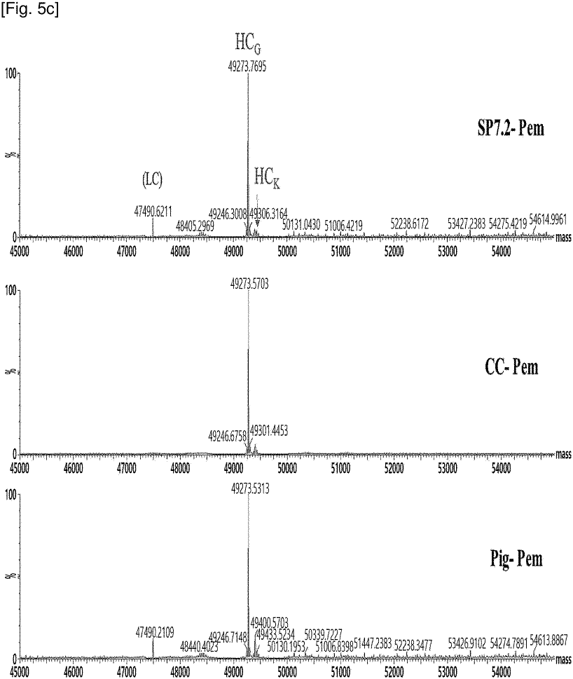

The mCherry sequences including the 5 types of signal peptides (Clus, Pig, Nuc, CC, and Proco) selected in Example 2 and the control SP7.2 were identically inserted into a specific site of the CHO genome to quantitatively compare the expression levels (FIGS. 4 and 5).

The insertion site was set at the Hprt Site, and a homology arm sequence and sgRNA sequence were designed with reference to J.S Lee et al., 2015, “Site-Specific integration in CHO cells mediated by CRISPR/Cas9 and homology-directed DNA repair pathway”, Sci. Rep., 5.

In the case of the 5′ homology arm, PCR was performed using primers containing Bg1 II and Nrul enzyme sites along with the CHO—S genome as a template. Thereafter, it was cloned into a pcDNA3.1(+) vector digested with Bg1II/Nrul.

In the case of 3′ homology arm, PCR was conducted using each primer containing Sall site along with the CHO—S genome as a template, and then Sall single cut was made along with the vector inserted with 5′ homology arm, and it was cloned into the downstream of the NeoR gene (pcDNA3.1_hprt).

In order to confirm only those that have been undergone homology recombination and inserted into the genome, Cmy-GFP-BHG pA fragments were constructed in the upstream region of the 5′ homology arm and inserted into Spel and Bg1II (pcDNA3.1_G_hprt) for double selection.

In the case of the GFP fragment, it was first inserted into the MCS of the pcDNA3.1(+) Vector with Ncol/Xbal, and then PCR was performed using primers containing Spel and Bg1II restriction sites. Thereafter, Spel was inserted using the Bg1II site into the vector (pcDNA_hprt) containing the homology.

Using the completed pcDNA3.1_G_hprt Vector, the mCherry gene sequences containing the signal peptide sequence was cut with Kpnl and Xhol and inserted into the MCS region.

As a result, the pcDNA3.1-based expression vector containing the expression cassette in the form of CMV-EGFP-pA-5′ Hprt Homology Arm-CMV-signal peptide candidate-mCherry-BGH pA-NeoR selection marker cassette-3′Hprt homology arm was constructed.

### 3-2. Site-Specific Integration

Knock-in was performed using CRISPR-Cas9 in order to insert the 6 types of vectors for site specific integration into the Hprt Site in the CHO-S genome. 240 ng of sgRNA, 1,250 ng of cas9 protein, and 1 µg of donor vector were independently mixed with Nucleofector solution to prepare 50 µL mixture.

CHO-S 1×10⁶ cells were first dissolved in 50 µL of Nucleofector and then mixed with the previously prepared mixture, and subsequently, the final 100 µL of the mixture was subjected to electroporation.

After performing the electroporation, the mixture was mixed with 0.5 mL of the medium, added to 2.5 mL of the medium, and cultured in a 6 well-plate at 36.5° C. and 5% CO₂.

After 2 days, selection was performed in the CD CHO media containing Zeneticin (0.5 mg/L), and then the cells were sub-cultured until 90% of viability was recovered.

After 90% of viability was recovered, 4 mL of the cells were cultured in duplicate in a 6 well-plate at a concentration of 3×10⁵ cells/mL, and the Vi-Cell and the medium fluorescence values (587 nm/610 nm) were measured every 2 to 3 days (FIGS. 5).

As a result, it was confirmed that the CC, Clus, and Pig secretory peptides showed higher expression than that of the control SP7.2.

### Example 4. Expression of Anti PD-1 Antibody and Mass Analysis

### 4-1. Preparation of Expression Vectors for Production of Anti PD-1 Antibody

After comparing the expression ability of signal peptides through site specific integration, anti-PD-1 antibodies fused with the 4 types of signal peptides (CC, Pig, Clus, and SP7.2) including CC, Clus, and Pig showing high expression were expressed. The Pembrolizumab (Keytruda®) antibody sequence was used as the target protein.

DNA sequences corresponding to the amino acid sequences of the light chain and the heavy chain were synthesized, and subsequently, sequences fused with each of the signal peptide sequences were produced through overlap PCR.

In the case of the light chain, the amino acid sequences were restricted with BamHI and Xhol, and in the case of the heavy chain, the amino acid sequences were restricted with AscI and Notl, and then the antibodies were inserted into the pTz-D1G1 vector, a variant of pcDNA3.1 (+) (including the promoter of KR Patent No. 10-1038126B1).

### pCB SP7.2 Pem

‘(N-terminal) - [BamHI Restriction Site - Signal Peptide (SEQ ID NO: 33) -Pem Light Chain (SEQ ID NO: 58) - Xhol Restriction Site] - (C-terminal)’ / ‘(N-terminal) - [AscI Restriction Site - Signal Peptide (SEQ ID NO: 33) - Pem Heavy Chain (SEQ ID NO: 59) - Notl Restriction Site] - (C-terminal)’

### pCB Clus Pem

‘(N-terminal) - [BamHI Restriction Site - Signal Peptide (SEQ ID NO: 4) - Pem Light Chain (SEQ ID NO: 58) - Xhol Restriction Site] - (C-terminal)’ / ‘(N-terminal) -[AscI Restriction Site - Signal Peptide (SEQ ID NO: 4) - Pem Heavy Chain (SEQ ID NO: 59) - Notl Restriction Site] - (C-terminal)’

### pCB CC Pem

‘(N-terminal) - [BamHI Restriction Site - Signal Peptide (SEQ ID NO: 2) - Pem Light Chain (SEQ ID NO: 58) - Xhol Restriction Site] - (C-terminal)’ / ‘(N-terminal) -[AscI Restriction Site - Signal Peptide (SEQ ID NO: 2) - Pem Heavy Chain (SEQ ID NO: 59) - Notl Restriction Site] - (C-terminal)’

### pCB Pig Pem

‘(N-terminal) - [BamHI Restriction Site - Signal Peptide (SEQ ID NO: 5) - Pem Light Chain (SEQ ID NO: 58) - Xhol Restriction Site] - (C-terminal)’ / ‘(N-terminal) -[AscI Restriction Site - Signal Peptide (SEQ ID NO: 5) - Pem Heavy Chain (SEQ ID NO: 59) - Notl Restriction Site] - (C-terminal)’

### 4-2. Expression of Anti-PD1 Antibody

The prepared recombinant expression vectors pCB-SP7.2-Pem, pCB-Clus-Pem, pCB-Pig-Pem and pCB-CC-Pem were introduced into ExpiCHO-S™ cells (Thermo Fisher Scientific), and cultured in the ExpiCHO expression medium (Thermo Fisher Scientific; 30 mL) for 12 days (Fed-Batch Culture; Day 1 & Day 5 Feeding) to express the fusion polypeptide (i.e., Pembrolizumab).

### 4-3. Purification of Anti-PD1 Antibody and Mass Analysis

The fusion polypeptide produced through the expression of the recombinant vectors was purified by ProteinA. Specifically, the recovered culture solution was filtered with a 0.22 µm filter, and then a column packed with ProteinA resin (Hitrap MSS, GE Healthcare, 11-0034-93) was mounted on AKTA™ Avant25 (GE Healthcare Life Sciences) and a PBS buffer was flowed through to equilibrate the column.

After the filtered culture solution was injected into a column, a PBS buffer was flowed through again to wash the column. After washing of the column was completed, an elution buffer (citrate buffer, pH 3.5) was flowed through the column to elute the target protein. The eluate was concentrated using the Amicon Ultra filter device (MWCO 30K, Merck) and a centrifuge. After the concentration was performed, buffer exchange was performed with PBS.

Quantitative analysis of the fusion polypeptide was performed by measuring the absorbance at 280 nm and 340 nm using a UV spectrophotometer (G113A, Agilent Technologies), and employing the following calculation equation. The extinction coefficient of each material was a value theoretically calculated using the amino acid sequence (1.404).

$$\begin{matrix}\text{Protein Concentration} \left(\text{mg} / \text{mL}\right) = \\ \frac{\text{Absorbance} \left({\text{A}}_{280 \text{nm}} - {\text{A}}_{340 \text{nm}}\right)}{* \text{Extinction Coefficient}} \times \text{Dilution Factor}\end{matrix}$$

(*Extinction Coefficient (0.1 %): It is a theoretical absorbance at 280 nm under assumption that the protein concentration is 0.1% (1 g/L), and all cysteines on the primary sequence are oxidized to form a disulfide bond. Calculated via ProtParam tool (https://web.expasy.org/protparam/)

The purified target proteins were used to confirm the presence of mis-cleavage of signal peptides at the N-terminus of the proteins using Q-TOF MS (FIG. 6). After dilution to a concentration of 1 mg/mL, the proteins were treated with PNGaseF, followed by 6 M Guanidine and DTT, and then loaded onto Q-TOF MS (RMM-MT-001: ACQUITY UPLC+Q-TOF SYNAPT G2 (Waters)).

As a result, it was confirmed that 100% cleavage was observed at the predicted cleavage sites.

Based on the results, the signal peptide, which is the CHO cell-derived protein secretory factor of the present invention, improves productivity by increasing the expression level of the recombinant proteins, and by confirming through mass analysis that 100% cleavage was observed at the predicted cleavage sites, it implies that the signal peptide of the present invention can be a powerful genetic tool which can solve the mis-cleavage, which is the problem of the existing protein secretory factors.

While the present invention has been described with reference to the particular illustrative embodiments, it will be understood by those skilled in the art to which the present invention pertains that the present invention may be embodied in other specific forms without departing from the technical spirit or essential characteristics of the present invention. Therefore, the embodiments described above are considered to be illustrative in all respects and not restrictive. Furthermore, the scope of the present invention is defined by the appended claims rather than the detailed description, and it should be understood that all modifications or variations derived from the meanings and scope of the present invention and equivalents thereof are included in the scope of the appended claims.

1. A protein secretory factor consisting of an amino acid sequence of SEQ ID NO: 1, SEQ ID NO: 2, or SEQ ID NO: 3.

2. The protein secretory factor of claim 1, wherein the protein is an endogenous protein or a foreign protein.

3. An expression cassette in which a nucleic acid sequence encoding a protein secretory factor consisting of an amino acid sequence of SEQ ID NO: 1, SEQ ID NO: 2, SEQ ID NO: 3, SEQ ID NO: 4, or SEQ ID NO: 5; and a gene encoding a target protein are operably linked.

4. The expression cassette of claim 3, wherein the target protein is selected from the group consisting of antibody, antibody fragment (Fab or ScFv), fusion protein, protein scaffold, human growth hormone, serum protein, immunoglobulin, cytokine, α-, β- or γ-interferon, granulocyte-macrophage colony-stimulating factor (GM-CSF), platelet-derived growth factor (PDGF), phospholipase-activating protein (PLAP), insulin, tumor necrosis factor (TNF), growth factor, hormone, calcitonin, calcitonin gene-related peptide (CGRP), enkephalin, somatomedin, erythropoietin, hypothalamic-releasing factor, growth differentiation factor, cell adhesion protein, prolactin, chorionic gonadotropin, tissue plasminogen activator, growth hormone-releasing peptide (GHPR), thymic humoral factor (THF), asparaginase, arginase, arginine deaminase, adenosine deaminase, peroxide dismutase, endotoxinase, catalase, chymotrypsin, lipase, uricase, adenosine diphosphatase, tyrosinase, bilirubin oxidase, glucose oxidase, glucodase, galactosidase, glucocerebrosidase, and glucuronidase.

5. The expression cassette of claim 3, wherein the nucleic acid sequence encoding the protein secretory factor consisting of the amino acid sequence of SEQ ID NO: 1, SEQ ID NO: 2, SEQ ID NO: 3, SEQ ID NO: 4, or SEQ ID NO: 5 consists of the nucleic acid sequence of SEQ ID NO: 11, SEQ ID NO: 12, SEQ ID NO: 13, SEQ ID NO: 14, or SEQ ID NO: 15.

6. The expression cassette of claim 3, wherein the expression cassette further comprises a nucleic acid sequence encoding any one of protein secretory factors consisting of an amino acid sequence of SEQ ID NO: 6, SEQ ID NO: 7, SEQ ID NO: 8, SEQ ID NO: 9, and SEQ ID NO: 10.

7. The expression cassette of claim 3, wherein the expression cassette expresses a target protein in which no additional amino acids have been added, from which the nucleic acid sequence encoding the secretory factor has been removed, when expressed in a cell, as it is.

8. An expression vector for secreting a target protein, comprising the expression cassette of claim 3.

9. The expression vector of claim 8, wherein the target protein is selected from the group consisting of antibody, antibody fragment (Fab or ScFv), fusion protein, protein scaffold, human growth hormone, serum protein, immunoglobulin, cytokine, α-, β- or γ-interferon, granulocyte-macrophage colony-stimulating factor (GM-CSF), platelet-derived growth factor (PDGF), phospholipase-activating protein (PLAP), insulin, tumor necrosis factor (TNF), growth factor, hormone, calcitonin, calcitonin gene-related peptide (CGRP), enkephalin, somatomedin, erythropoietin, hypothalamic-releasing factor, growth differentiation factor, cell adhesion protein, prolactin, chorionic gonadotropin, tissue plasminogen activator, growth hormone releasing peptide (GHPR), thymic humoral factor (THF), asparaginase, arginase, arginine deaminase, adenosine deaminase, peroxide dismutase, endotoxinase, catalase, chymotrypsin, lipase, uricase, adenosine diphosphatase, tyrosinase, bilirubin oxidase, glucose oxidase, glucodase, galactosidase, glucocerebrosidase, and glucuronidase.

10. The expression vector of claim 8, wherein the nucleic acid sequence encoding the protein secretory factor consisting of the amino acid sequence of SEQ ID NO: 1, SEQ ID NO: 2, SEQ ID NO: 3, SEQ ID NO: 4, or SEQ ID NO: 5 consists of the nucleic acid sequence of SEQ ID NO: 11, SEQ ID NO: 12, SEQ ID NO: 13, SEQ ID NO: 14, or SEQ ID NO: 15.

11. The expression vector of claim 8, wherein the expression vector further comprises a nucleic acid sequence encoding any one of protein secretory factors consisting of an amino acid sequence of SEQ ID NO: 6, SEQ ID NO: 7, SEQ ID NO: 8, SEQ ID NO: 9, and SEQ ID NO: 10.

12. The expression vector of claim 8, wherein the expression vector expresses a target protein in which no additional amino acids have been added, from which the nucleic acid sequence encoding the secretory factor has been removed, when expressed in a cell, as it is.

13. A transformed cell in which the expression vector of claim 8 is introduced into a host cell.

14. The transformed cell of claim 13, wherein the host cell is a Chinese hamster ovary cell (CHO cell).

15. A method for producing a target protein, comprising:
i) culturing a transformed cell comprising an expression vector for secreting a target protein, which includes an expression cassette in which a nucleic acid sequence encoding a protein secretory factor consisting of an amino acid sequence of SEQ ID NO: 1, SEQ ID NO: 2, SEQ ID NO: 3, SEQ ID NO: 4, or SEQ ID NO: 5; and a gene encoding a target protein are operably linked; and
ii) recovering the target protein from the culture medium or culture supernatant of the cultured cells.

16. The method of claim 15, further comprising purifying the recovered target protein.

17. The method of claim 15, wherein the host cell is a Chinese hamster ovary cell (CHO cell).

18. The method of claim 15, wherein the protein secretory factor consisting of the amino acid sequence of SEQ ID NO: 1, SEQ ID NO: 2, SEQ ID NO: 3, SEQ ID NO: 4, or SEQ ID NO: 5 is cleaved at the N-terminal cleavage site of the target protein.

19. The method of claim 15, wherein the target protein is a target protein itself in which no additional amino acids have been added, from which the nucleic acid sequence encoding the secretory factor has been removed.
